(19) United States
(12) Patent Application Publication
(10) Pub. No.: US 2025/0026773 A1
(43) Pub. Date: Jan. 23, 2025

(54) MONO-P-TOLUENESULFONATE OF AXL KINASE INHIBITOR AND CRYSTAL FORM THEREOF

(71) Applicant: NANJING CHIA TAI TIANQING PHARMACEUTICAL CO., LTD., Nanjing (CN)

(72) Inventors: Changyou MA, Nanjing (CN); Linlin ZHANG, Nanjing (CN); Haiwei FENG, Nanjing (CN); Tingli ZHAO, Nanjing (CN); Jian WU, Nanjing (CN); Dan XU, Nanjing (CN); Chunxia ZHU, Nanjing (CN); Zhoushan TIAN, Nanjing (CN)

(21) Appl. No.: 18/713,611

(22) PCT Filed: Nov. 25, 2022
(86) PCT No.: PCT/CN2022/134410

§ 371 Date: May 24, 2024

(30) Foreign Application Priority Data
Nov. 26, 2021 (CN) 202111425629.7

(51) Int. Cl. C07F 9/6558; A61K 31/675; C07C 309/30

(52) U.S. Cl. CPC C07F 9/65583; A61K 31/675; C07C 309/30

(57) ABSTRACT

The present invention provides a mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethyl phosphine oxide, and a hydrate and a crystal form thereof. The crystal form of p-toluenesulfonate has a good stability, is easy to process, and has a high solubility.

### TECHNICAL FIELD

The present invention belongs to the field of medical technology. The compound is an AXL kinase inhibitor, and specifically relates to p-toluenesulfonate of the AXL inhibitor and crystal form thereof.

### BACKGROUND

Receptor tyrosine kinases (RTKs) are multidomain transmembrane proteins that serve as sensors for extracellular ligands. Ligand-receptor binding induces receptor dimerization and activation of its intracellular kinase domain, which in turn leads to the recruitment, phosphorylation, and activation of multiple downstream signaling cascades (Robinson, D R et al., Oncogene, 19:5548-5557, 2000). To date, 58 RTKs have been identified in the human genome, which regulate a variety of cellular processes, including cell survival, growth, differentiation, proliferation, adhesion, and motility (Segaliny, Al et al., J. Bone Oncol, 4:1-12, 2015).

AXL (also known as UFO, ARK and Tyro7) belongs to the TAM family of receptor tyrosine kinases, which also includes Mer and Tyro3. Among them, AXL and Tyro3 have the most similar gene structures, while AXL and Mer have the most similar amino acid sequences of tyrosine kinase domains. Like other receptor tyrosine kinases (RTKs), the structure of the TAM family consists of an extracellular domain, a transmembrane domain, and a conserved intracellular kinase domain. The extracellular domain of AXL has a unique structure that juxtaposes immunoglobulin and type III fibronectin repeating units and is reminiscent of that of neutral cell adhesion molecules. TAM family members have a common ligand-growth arrest specific protein 6 (Gas6), which can bind to all TAM receptor tyrosine kinases. After AXL binds to Gas6, it will lead to receptor dimerization and AXL autophosphorylation, thereby activating multiple downstream signal transduction pathways and participating in multiple processes of tumorigenesis (Linger, R. M et al., Ther. Targets, 14 (10), 1073-1090, 2010; Rescigno, J. et al., Oncogene, 6(10), 1909-1913, 1991).

AXL is widely expressed in normal human tissues, such as monocytes, macrophages, platelets, endothelial cells, cerebellum, heart, skeletal muscle, liver and kidney, etc. Among them, myocardium and skeletal muscle have the highest expression, and bone marrow CD34+ cells and stromal cells also have higher expression, normal lymphoid tissue has low expression (Wu Y M, Robinson D R, Kung H J, Cancer Res, 64 (20), 7311-7320, 2004; Hung B I et al., DNA Cell Biol, 22 (8), 533-540, 2003). In studies of many cancer cells, it has been found that the AXL gene is overexpressed or ectopically expressed in hematopoietic cells, stromal cells, and endothelial cells. The overexpression of AXL kinase is particularly prominent in various types of leukemias and most solid tumors. By inhibiting AXL receptor tyrosine kinase, the pro-survival signals of tumor cells can be reduced, the invasion ability of tumors can be blocked, and the sensitivity of targeted drug therapy and chemotherapy can be increased. Therefore, finding effective AXL inhibitors is an important direction in the current research and development of tumor-targeted medicaments.

### SUMMARY OF THE INVENTION

In one aspect, the present invention provides a mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or a hydrate thereof.

Further, the mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof, described herein, has the structure as shown in formula I:

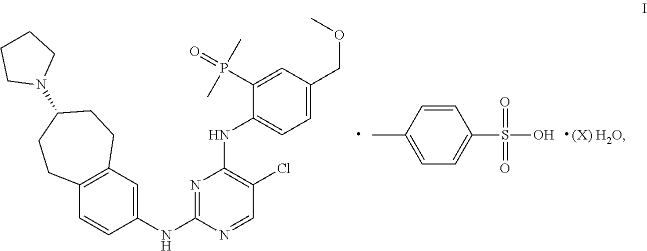

wherein X=0˜2.

Further, X=0˜1 or 1.5.

Further, X is 0, 0.25, 0.5, 0.7, 1, 1.25, 1.5 or 1.75.

In some typical embodiments, X is 0.

In some typical embodiments, X is 0.7.

In some typical embodiments, X is 1.

In some typical embodiments, X is 1.5.

Further, the present invention provides a hydrate of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide.

In some typical embodiments, the present invention provides a monohydrate of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, the specific structure is shown in formula II,

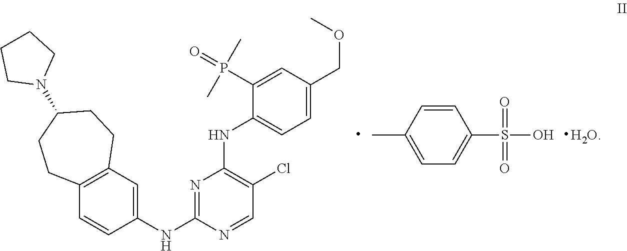

In some typical embodiments, the present invention provides a sesquihydrate of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, the specific structure is shown in formula III,

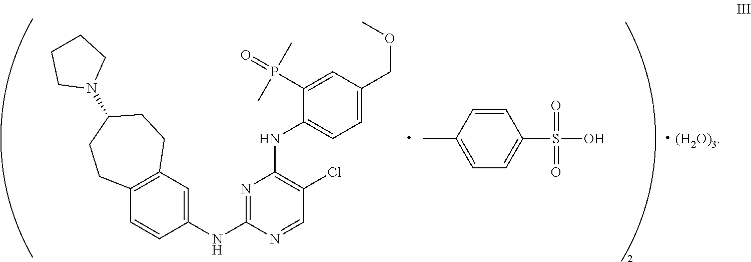

Further, the present invention provides a crystal form of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof.

Further, the present invention provides a crystal form of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof, the X-ray powder diffraction pattern has diffraction peaks at 2θ of 6.0°±0.2°, 6.3°±0.2°, 10.5°±0.2°, 13.2°±0.2° and 21.8°±0.2°.

In some typical embodiments, the X-ray powder diffraction pattern of the crystal has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 21.8°±0.2° and 22.6°±0.2°.

In some typical embodiments, the X-ray powder diffraction pattern of the crystal has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 18.7°±0.2°, 19.4°±0.2°, 19.7°±0.2°, 21.8°±0.2°, 22.6°±0.2° and 29.4°±0.2°.

In some more typical embodiments, the X-ray powder diffraction pattern of the crystal has diffraction peaks at 2θ of 6.0°±0.2°, 6.3°±0.2°, 10.5°±0.2°, 11.5°±0.2°, 12.3°±0.2°, 12.4°±0.2°, 12.6°±0.2°, 13.2°±0.2°, 14.1°±0.2°, 15.2°±0.2°, 15.9°±0.2°, 16.7°±0.2°, 17.1°±0.2°, 18.0°±0.2°, 18.6°±0.2°, 18.7°±0.2°, 19.0°±0.2°, 19.4°±0.2°, 19.7°±0.2°, 21.1°±0.2°, 21.8°±0.2°, 22.6°±0.2°, 23.3°±0.2°, 23.7°±0.2°, 24.1°±0.2°, 24.4°±0.2°, 24.7°±0.2°, 25.1°±0.2°, 26.2°±0.2°, 26.6°±0.2°, 27.0°±0.2°, 27.7°±0.2°, 28.0°±0.2°, 28.3°±0.2°, 28.7°±0.2°, 28.8°±0.2°, 29.4°±0.2°, 30.2°±0.2° and 33.9°±0.2°.

In some embodiments, the crystal has an endothermic peak at an onset temperature of 195±5° C.˜205±5° C. in a thermal analysis diagram measured by differential scanning calorimetry.

In some embodiments, the crystal has an endothermic peak at an onset temperature of 198±5° C.˜203±5° C. in a thermal analysis diagram measured by differential scanning calorimetry.

In some embodiments, the crystal has an endothermic peak at an onset temperature of 200±5° C. in a thermal analysis diagram measured by differential scanning calorimetry.

Further, the present invention provides a crystal form of the compound of formula I, wherein X=0˜1, the X-ray powder diffraction pattern has diffraction peaks at 2θ of 6.0°±0.2°, 6.3°±0.2°, 10.5°±0.2°, 13.2°±0.2° and 21.8°±0.2°.

In some typical embodiments, the X-ray powder diffraction pattern of the crystal has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 21.8°±0.2° and 22.6°±0.2°.

In some typical embodiments, the X-ray powder diffraction pattern of the crystal has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 18.7°±0.2°, 19.4°±0.2°, 19.7°±0.2°, 21.8°±0.2°, 22.6°±0.2° and 29.4°±0.2°.

In some more typical embodiments, the X-ray powder diffraction pattern of the crystal has diffraction peaks at 2θ of 6.0°±0.2°, 6.3°±0.2°, 10.5°±0.2°, 11.5°±0.2°, 12.3°±0.2°, 12.4°±0.2°, 12.6°±0.2°, 13.2°±0.2°, 14.1°±0.2°, 15.2°±0.2°, 15.9°±0.2°, 16.7°±0.2°, 17.1°±0.2°, 18.0°±0.2°, 18.6°±0.2°, 18.7°±0.2°, 19.0°±0.2°, 19.4°±0.2°, 19.7°±0.2°, 21.1°±0.2°, 21.8°±0.2°, 22.6°±0.2°, 23.3°±0.2°, 23.7°±0.2°, 24.1°±0.2°, 24.4°±0.2°, 24.7°±0.2°, 25.1°±0.2°, 26.2°±0.2°, 26.6°±0.2°, 27.0°±0.2°, 27.7°±0.2°, 28.0°±0.2°, 28.3°±0.2°, 28.7°±0.2°, 28.8°±0.2°, 29.4°±0.2°, 30.2°±0.2° and 33.9°±0.2°.

In some embodiments, the crystal has an endothermic peak at an onset temperature of 195±5° C.˜205±5° C. in a thermal analysis diagram measured by differential scanning calorimetry.

In some embodiments, the crystal has an endothermic peak at an onset temperature of 198±5° C.˜203±5° C. in a thermal analysis diagram measured by differential scanning calorimetry.

In some more typical embodiments, the crystal has an endothermic peak at an onset temperature of 200° C.±5° C. in a thermal analysis diagram measured by differential scanning calorimetry.

Further, the present invention provides a crystal form B of mono-p-toluenesulfonate monohydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, the X-ray powder diffraction pattern has diffraction peaks at 2θ of 6.0°±0.2°, 6.3°±0.2°, 10.5°±0.2°, 13.2°±0.2° and 21.8°±0.2°.

In some embodiments, the X-ray powder diffraction pattern of the crystal form B has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 21.8°±0.2° and 22.6°±0.2°.

In some embodiments, the X-ray powder diffraction pattern of the crystal form B has diffraction peaks at 2° of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 18.7°±0.2°, 19.4°±0.2°, 19.7°±0.2°, 21.8°±0.2°, 22.6°±0.2° and 29.4°±0.2.

In some embodiments, the X-ray powder diffraction pattern of the crystal form B has diffraction peaks at 2θ of 6.0°±0.2°, 6.3°±0.2°, 10.5°±0.2°, 11.5°±0.2°, 12.3°±0.2°, 12.4°±0.2°, 12.6°±0.2°, 13.2°±0.2°, 14.1°±0.2°, 15.2°±0.2°, 15.9°±0.2°, 16.7°±0.2°, 17.1°±0.2°, 18.0°±0.2°, 18.6°±0.2°, 18.7°±0.2°, 19.0°±0.2°, 19.4°±0.2°, 19.7°±0.2°, 21.1°±0.2°, 21.8°±0.2°, 22.6°±0.2°, 23.3°±0.2°, 23.7°±0.2°, 24.1°±0.2°, 24.4°±0.2°, 24.7°±0.2°, 25.1°±0.2°, 26.2°±0.2°, 26.6°±0.2°, 27.0°±0.2°, 27.7°±0.2°, 28.0°±0.2°, 28.3°±0.2°, 28.7°±0.2°, 28.8°±0.2°, 29.4°±0.2°, 30.2°±0.2° and 33.9°±0.2°.

In some more typical embodiments, the X-ray powder diffraction of the crystal form B expressed in an angle of 2θ has the data described in the following table:

TABLE 1
X-ray powder diffraction pattern data of crystal form B

| 2θ (°) | Counts | I/I₀ (%) |
|---|---|---|
| 6.0 | 1772 | 100.0 |
| 6.3 | 1424 | 80.3 |
| 7.7 | 36 | 2.0 |
| 8.1 | 26 | 1.4 |
| 9.2 | 12 | 0.7 |
| 9.7 | 16 | 0.9 |
| 10.5 | 1448 | 81.7 |
| 11.5 | 970 | 54.8 |
| 12.3 | 215 | 12.1 |
| 12.4 | 133 | 7.5 |
| 12.6 | 169 | 9.5 |
| 13.2 | 1033 | 58.3 |
| 13.6 | 53 | 3.0 |
| 14.1 | 190 | 10.7 |
| 15.2 | 804 | 45.4 |
| 15.9 | 281 | 15.9 |
| 16.7 | 323 | 18.2 |
| 17.1 | 292 | 16.5 |
| 18.0 | 974 | 54.9 |
| 18.6 | 947 | 53.4 |
| 18.7 | 449 | 25.3 |
| 19.0 | 354 | 20.0 |
| 19.4 | 764 | 43.1 |
| 19.7 | 487 | 27.5 |
| 20.6 | 50 | 2.8 |
| 21.1 | 239 | 13.5 |
| 21.8 | 1152 | 65.0 |
| 22.6 | 844 | 47.6 |
| 23.4 | 106 | 6.0 |
| 23.7 | 232 | 13.1 |
| 24.1 | 184 | 10.4 |
| 24.4 | 181 | 10.2 |
| 24.7 | 274 | 15.5 |
| 25.1 | 227 | 12.8 |
| 25.6 | 71 | 4.0 |
| 26.3 | 177 | 10.0 |
| 26.6 | 223 | 12.6 |
| 27.0 | 371 | 20.9 |
| 27.7 | 249 | 14.1 |
| 28.0 | 143 | 8.1 |
| 28.3 | 226 | 12.8 |
| 28.7 | 109 | 6.2 |
| 28.8 | 303 | 17.1 |
| 29.4 | 459 | 25.9 |
| 30.2 | 281 | 15.8 |
| 32.0 | 81 | 4.5 |
| 33.1 | 68 | 3.9 |
| 33.9 | 152 | 8.6 |
| 35.1 | 58 | 3.3 |
| 35.7 | 44 | 2.5 |

In some embodiments, the X-ray powder diffraction of the crystal form B expressed in an angle of 2θ has a pattern as shown in FIG. 1.

In some embodiments, the crystal form B has an endothermic peak at an onset temperature of 85±5° C. to 95±5° C. and an endothermic peak at an onset temperature of 195±5° C.˜205±5° C. in a thermal analysis diagram measured by differential scanning calorimetry.

In some embodiments, the crystal form B has an endothermic peak at an onset temperature of 88±5° C. to 93±5° C. and an endothermic peak at an onset temperature of 198±5° C. to 203±5° C. in a thermal analysis diagram measured by differential scanning calorimetry.

In some embodiments, the crystal form B has an endothermic peak at an onset temperature of 90±5° C. and an endothermic peak at an onset temperature of 200±5° C. in a thermal analysis diagram measured by differential scanning calorimetry.

In some embodiments, the crystal form B has a pattern as shown in FIG. 2 in a thermal analysis diagram measured by differential scanning calorimetry.

In some embodiments, a spectrum of the crystal form B that is obtained by attenuated total reflectance Fourier transform infrared spectroscopy has the following absorption bands expressed in reciprocals of wavelengths (cm⁻¹): 432±2, 471±2, 552±2, 570±2, 711±2, 747±2, 779±2, 818±2, 836±2, 863±2, 932±2, 966±2, 1295±2, 1318±2, 2635±2, 2721±2, 2927±2, 3005±2, 3110±2, 3185±2, 3256±2 and 3556±2.

In some embodiments, a spectrum of the crystal form B that is obtained by attenuated total reflectance Fourier transform infrared spectroscopy has the following absorption bands expressed in reciprocals of wavelengths (cm⁻¹): 432±2, 471±2, 497±2, 552±2, 570±2, 681±2, 711±2, 747±2, 779±2, 818±2, 836±2, 863±2, 932±2, 966±2, 1008±2, 1030±2, 1096±2, 1121±2, 1159±2, 1229±2, 1295±2, 1318±2, 1374±2, 1413±2, 1456±2, 1512±2, 1527±2, 1571±2, 1609±2, 2635±2, 2721±2, 2927±2, 3005±2, 3110±2, 3185±2, 3256±2 and 3556±2.

In some embodiments, a spectrum of the crystal form B that is obtained by Fourier transform Raman spectroscopy has the following absorption bands expressed in reciprocals of wavelengths (cm⁻¹): 1609±2, 1572±2, 1553±2, 1535±2, 1508±2, 1494±2, 1476±2, 1457±2, 1420±2, 1374±2, 1344±2, 1331±2, 1285±2, 1262±2, 1228±2, 1213±2, 1173±2, 1147±2, 1135±2, 1099±2, 1059±2, 1029±2, 1115±2, 1006±2, 970±2, 965±2, 819±2, 799±2, 744±2, 733±2, 678±2, 658±2, 634±2, 613±2, 570±2, 534±2, 497±2, 471±2, 448±2, 425±2, 393±2, 369±2, 313±2, 287±2, 262±2, 241±2, 217±2, 177±2, 156±2, 104±2 and 82±2.

In some embodiments, the crystal form B lost 2.4% of its weight in the temperature range of 25° C.-150° C.

In some embodiments, the crystal form B has a TGA pattern as shown in FIG. 3.

In some more exemplary embodiments, the crystal form B has single crystal parameters and structural data as described in the following table:

TABLE 2
crystal parameters and structural data of crystal form B
Analytical data

| Molecular formula | C₂₉H₃₈ClN₅O₂P•C₇H₇O₃S•H₂O |
|---|---|
| Molecular weight | 744.28 |
| Crystal system | Orthorhombic |
| Space group | P2₁2₁2₁ |
| Cell parameters | a = 7.9891(3)Å, α = 90° |
| | b = 15.7648(7)Å, β = 90° |
| | c = 29.3381(14)Å, γ = 90° |
| Crystal axis ratio | a/b = 0.5068, b/c = 0.5367, c/a = 3.6723 |
| Z | 4 |
| Unit cell volume | 3695.0(3)Å³ |
| Theoretical density | 1.338 Mg/m³ |
| R₁ | 0.050 |
| WR₂ | 0.114 |
| GOOF═S | 1.03 |
| R₍ᵢₙₜ₎ | 0.082 |
| Flackparameter | 0.019(19) |

Further, the present invention provides a crystal form C of mono-p-toluenesulfonate sesquihydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, the specific structure is shown in formula III:

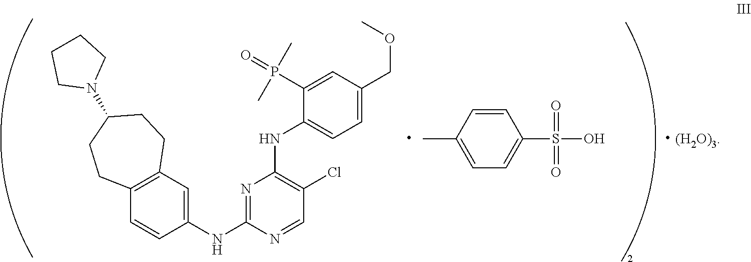

The X-ray powder diffraction pattern has diffraction peaks at 2θ of 13.4°±0.2°, 18.4°±0.2°, 19.3°±0.2°, 19.8°±0.2° and 21.8°±0.2°.

In some embodiments, the X-ray powder diffraction pattern of the crystal form C has diffraction peaks at 2θ of 12.1°±0.2°, 13.4°±0.2°, 15.0°±0.2°, 16.9°±0.2°, 18.4°±0.2°, 19.3°±0.2°, 19.8°±0.2°, 21.8°±0.2°, 23.6°±0.2° and 24.3°±0.2°.

In some embodiments, the X-ray powder diffraction pattern of the crystal form C has diffraction peaks at 2θ of 6°±0.2°, 10.8°±0.2°, 12.1°±0.2°, 13.4°±0.2°, 15.0°±0.2°, 16.9°±0.2°, 18.4°±0.2°, 19.0°±0.2°, 19.3°±0.2°, 19.8°±0.2°, 20.9°±0.2°, 21.8°±0.2°, 23.2°±0.2°, 23.6°±0.2°, 24.3°±0.2°, 25.5°±0.2° and 26.6°±0.2°.

In some more exemplary embodiments, the 20 of the X-ray powder diffraction pattern of the crystal form C is detailed in the following table:

TABLE 3
X-ray powder diffraction pattern data of crystal form C

| 2θ (°) | Counts | I/I₀ (%) |
|---|---|---|
| 6.0 | 105 | 22.2 |
| 10.8 | 60 | 12.8 |
| 12.1 | 227 | 48.2 |
| 13.4 | 322 | 68.5 |
| 15.0 | 220 | 46.7 |
| 16.9 | 221 | 46.9 |
| 18.4 | 471 | 100.0 |
| 19.0 | 176 | 37.5 |
| 19.3 | 238 | 50.5 |
| 19.8 | 306 | 65.0 |
| 20.9 | 154 | 32.8 |
| 21.8 | 310 | 65.9 |
| 23.2 | 172 | 36.6 |
| 23.6 | 231 | 49.1 |
| 24.3 | 213 | 45.2 |
| 25.5 | 62 | 13.1 |
| 26.6 | 103 | 21.8 |
| 27.7 | 38 | 8.0 |
| 29.7 | 93 | 19.7 |

In some embodiments, X-ray powder diffraction of the crystal form C expressed in a 20 angle has a pattern as shown in FIG. 7.

In some more exemplary embodiments, the crystal form C has single crystal parameters and structural data as described in the following table:

TABLE 4
crystal parameters and structure data of crystal form C
Analytical data

| Molecular formula | 2(C₇H₇O₃S)•2(C₂₉H₃₈ClN₅O₂P)•3(H₂O) |
|---|---|
| Molecular weight | 1506.54 |
| Crystal system | Orthorhombic |
| Space group | P2₁2₁2₁ |
| Cell parameters | a = 8.4802(8)Å, α = 90° |
| | b = 15.1272(14)Å, β = 90° |
| | c = 29.429(3)Å, γ = 90° |
| Crystal axis ratio | a/b = 0.5606, b/c = 0.5140, c/a = 3.4703 |
| Z | 2 |
| Unit cell volume | 3775.2(7)Å³ |
| Theoretical density | 1.325 Mg/m³ |
| R₁ | 0.053 |
| WR₂ | 0.114 |
| GOOF═S | 1.06 |
| R₍ᵢₙₜ₎ | 0.041 |
| Flackparameter | 0.029(12) |

In another aspect, the present invention provides a crystal form A of the compound of formula IV, the X-ray powder diffraction pattern has diffraction peaks at 2θ of 7.6°±0.2°, 10.2°±0.2°, 17.6°±0.2°, 20.3°±0.2° and 20.9°±0.2°.

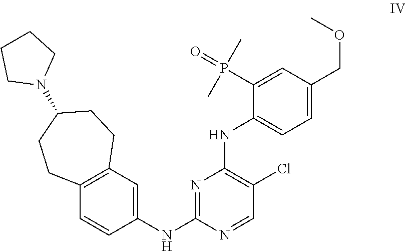 [SMILES: COCc1ccc(Nc2nc(Nc3ccc4c(c3)CC[C@@H](N3CCCC3)CC4)ncc2Cl)c(P(C)(C)=O)c1]

In some embodiments, the X-ray powder diffraction pattern of the crystal form A has diffraction peaks at 2θ of 4.1°±0.2°, 7.6°±0.2°, 10.2°±0.2°, 12.6°±0.2°, 13.0°±0.2°, 17.6°±0.2°, 19.7°±0.2°, 20.3°±0.2°, 20.9°±0.2° and 22.2°±0.2°.

In some embodiments, the X-ray powder diffraction pattern of the crystal form A has diffraction peaks at 2θ of 4.1°±0.2°, 5.6°±0.2°, 7.6°±0.2°, 10.2°±0.2°, 10.9°±0.2°, 12.6°±0.2°, 13.0°±0.2°, 15.2°±0.2°, 17.6°±0.2°, 19.7°±0.2°, 20.3°±0.2°, 20.9°±0.2°, 22.2°±0.2°, 23.2°±0.2°, 24.6°±0.2°, 27.0°±0.2°, 28.8°±0.2°, 37.0°±0.2° and 37.7°±0.2°.

In some more exemplary embodiments, the 2θ of the X-ray powder diffraction pattern of the crystal form A is detailed in the following table:

TABLE 5
X-ray powder diffraction pattern data of crystal form A

| 2θ (°) | Counts | I/I₀ (%) |
|---|---|---|
| 4.1 | 45 | 15.4 |
| 5.6 | 42 | 14.3 |
| 7.6 | 109 | 36.9 |
| 10.2 | 107 | 36.4 |
| 10.9 | 13 | 4.4 |
| 12.6 | 59 | 20.1 |
| 13.0 | 44 | 14.8 |
| 15.2 | 18 | 6.2 |
| 17.6 | 246 | 83.4 |
| 19.7 | 85 | 28.9 |
| 20.3 | 184 | 62.5 |
| 20.9 | 294 | 100.0 |
| 22.2 | 60 | 20.5 |
| 23.2 | 22 | 7.5 |
| 24.6 | 19 | 6.6 |
| 27.0 | 40 | 13.6 |
| 28.8 | 14 | 4.6 |
| 37.0 | 6 | 2.1 |
| 37.7 | 12 | 4.1 |

In some more exemplary embodiments, X-ray powder diffraction of the crystal form A expressed in 2θ angle has a pattern as shown in FIG. 9.

In another aspect, the present invention provides methods for preparing mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, or pharmaceutically acceptable hydrate thereof, comprising the step of salifying the compound of formula IV with p-toluenesulfonic acid.

In another aspect, the present invention provides methods for preparing the compound of formula I or crystal form thereof, comprising the step of salifying the compound of formula IV with p-toluenesulfonic acid.

In some embodiments, the crystal form of the compound of formula I can be prepared by mixing and stirring, gas-solid diffusion, solvent evaporation, or precipitation at reduced temperature.

In another aspect, the present invention provides methods for preparing the crystal form B by reacting the compound of formula IV with p-toluenesulfonic acid monohydrate in an alcoholic solvent and crystallizing in an anti-solvent.

In some embodiments, the alcohol solvent may be an alcohol solvent or a mixture of alcohol and water, the alcohol solvent is selected from one of methanol, ethanol and isopropanol, preferably ethanol or isopropanol.

In some embodiments, the anti-solvent is selected from ester solvent, ketone solvent or ether solvent, wherein the ketone solvent is acetone, 2-butanone or methyl isobutyl ketone, preferably acetone; the ether solvent is methyl tert-butyl ether or 1,4-dioxane, preferably methyl tert-butyl ether; and the ester solvent is selected from ethyl acetate, butyl acetate or isopropyl acetate, preferably isopropyl acetate.

In another aspect, the present invention provides a pharmaceutical composition comprising mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof.

In another aspect, the present invention provides a pharmaceutical composition comprising the compound of formula I.

In another aspect, the present invention provides a pharmaceutical composition comprising the compound of formula II.

In another aspect, the present invention provides a pharmaceutical composition comprising the compound of formula III.

In some embodiments, the pharmaceutical composition further comprises one or more pharmaceutically acceptable carriers.

In some embodiments, the pharmaceutical composition is solid pharmaceutical preparation suitable for oral administration, preferably tablets or capsules.

In another aspect, the present invention provides a crystal form composition comprising the crystal form A, wherein the crystal form A comprises more than 50% by weight of the weight of the crystal form composition; preferably more than 80% by weight; further preferably more than 90% by weight; further preferably more than 95% by weight; most preferably more than 98% by weight.

In another aspect, the present invention also provides a pharmaceutical composition comprises the crystal form A or crystal form composition thereof.

In some embodiments, the pharmaceutical composition further comprises one or more pharmaceutically acceptable carriers.

In some embodiments, the pharmaceutical composition is a solid pharmaceutical preparation suitable for oral administration, preferably tablets or capsules.

In another aspect, the present invention provides a crystal form composition comprising the crystal form B, wherein the crystal form B is more than 50% by weight of the weight of the crystal form composition; preferably more than 80% by weight; further preferably more than 90% by weight; further preferably more than 95% by weight; most preferably more than 98% by weight.

In another aspect, the present invention also provides a pharmaceutical composition comprising the crystal form B or crystal form composition.

In some embodiments, the pharmaceutical composition further comprises one or more pharmaceutically acceptable carriers.

In some embodiments, the pharmaceutical composition is a solid pharmaceutical preparation suitable for oral administration, preferably tablets or capsules.

In another aspect, the present invention provides a crystal form composition comprising the crystal form C, wherein the crystal form C comprises more than 50% by weight of the weight of the crystal form composition; preferably more than 80% by weight; further preferably more than 90% by weight; further preferably more than 95% by weight; most preferably more than 98% by weight.

In another aspect, the present invention also provides a pharmaceutical composition comprising the crystal form C or crystal form composition thereof.

In some embodiments, the pharmaceutical composition further comprises one or more pharmaceutically acceptable carriers.

In some embodiments, the pharmaceutical composition is a solid pharmaceutical preparation suitable for oral administration, preferably tablets or capsules.

In another aspect, the present invention also provides the compound of formula I or pharmaceutical composition thereof for use as medicaments.

In another aspect, the present invention also provides the compound of formula II or pharmaceutical composition thereof for use as medicaments.

In another aspect, the present invention also provides the compound of formula III or pharmaceutical composition thereof for use as medicaments.

In another aspect, the present invention also provides the crystal form A, crystal form composition thereof or pharmaceutical composition thereof for use as medicaments.

In another aspect, the present invention also provides the crystal form B, crystal form composition thereof or pharmaceutical composition thereof for use as medicaments.

In another aspect, the present invention also provides the crystal form C, crystal form composition thereof or pharmaceutical composition thereof for use as medicaments.

In another aspect, the present invention also provides the use of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, hydrate thereof and pharmaceutical composition thereof in the preparation of medicaments for the prevention and/or treatment of AXL kinase-mediated diseases or disease states.

In another aspect, the present invention also provides the use of the compound of formula I or pharmaceutical composition thereof in the preparation of medicaments for the prevention and/or treatment of AXL kinase-mediated diseases or disease states.

In another aspect, the present invention also provides the use of the compound of formula II or pharmaceutical composition thereof in the preparation of medicaments for the prevention and/or treatment of AXL kinase-mediated diseases or disease states.

In another aspect, the present invention also provides the use of the compound of formula III or pharmaceutical composition thereof in the preparation of medicaments for the prevention and/or treatment of AXL kinase-mediated diseases or disease states.

In another aspect, the present invention also provides the use of the crystal form A or pharmaceutical composition thereof in the preparation of medicaments for the prevention and/or treatment of AXL kinase-mediated diseases or disease states.

In another aspect, the present invention also provides the use of the crystal form B or pharmaceutical composition thereof in the preparation of medicaments for the prevention and/or treatment of AXL kinase-mediated diseases or disease states.

In another aspect, the present invention also provides the use of the crystal form C or pharmaceutical composition thereof in the preparation of medicaments for the prevention and/or treatment of AXL kinase-mediated diseases or disease states.

In another aspect, the present invention also provides the use of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof in the preparation of medicaments in the prevention and/or treatment of an AXL kinase-mediated disease or disease state.

In another aspect, the present invention also provides the use of the compound of formula I in the preparation of medicaments for the prevention and/or treatment of AXL kinase-mediated diseases or disease states.

In another aspect, the present invention also provides the use of the compound of formula II in the preparation of medicaments for the prevention and/or treatment of AXL kinase-mediated diseases or disease states.

In another aspect, the present invention also provides the use of the compound of formula III in the preparation of medicaments for the prevention and/or treatment of AXL kinase-mediated diseases or disease states.

In another aspect, the present invention also provides the use of the crystal form composition in the preparation of medicaments for the prevention and/or treatment of AXL kinase-mediated diseases or disease states.

In another aspect, the present invention also provides the use of the crystal form A, crystal form composition thereof or pharmaceutical composition thereof for the prevention and/or treatment of AXL kinase-mediated diseases or disease states.

In another aspect, the present invention also provides the use of the crystal form B, crystal form composition thereof or pharmaceutical composition thereof for the prevention and/or treatment of AXL kinase-mediated diseases or disease states.

In another aspect, the present invention also provides the use of the crystal form C, crystal form composition thereof or pharmaceutical composition thereof for the prevention and/or treatment of AXL kinase-mediated diseases or disease states.

In another aspect, the present invention also provides methods for preventing and/or treating AXL kinase-mediated diseases or disease states comprising administering to an individual in need thereof the mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, hydrate thereof, or pharmaceutical composition thereof.

In another aspect, the present invention also provides methods for preventing and/or treating AXL kinase-mediated diseases or disease states, it comprises administering to an individual in need thereof the compound of formula I of the present invention or a pharmaceutical composition thereof.

In another aspect, the present invention also provides methods for preventing and/or treating AXL kinase-mediated diseases or disease states, it comprises administering to an individual in need thereof the compound of formula II of the present invention or a pharmaceutical composition thereof.

In another aspect, the present invention also provides methods for preventing and/or treating AXL kinase-mediated diseases or disease states, it comprises administering to an individual in need thereof the compound of formula III of the present invention or pharmaceutical composition thereof.

In another aspect, the present invention also provides methods for preventing and/or treating AXL kinase-mediated diseases or disease states, it comprises administering the crystal form composition of the present invention to an individual in need thereof.

In another aspect, the present invention also provides methods for preventing and/or treating AXL kinase-mediated diseases or disease states, it comprises administering the crystal form A of the present invention or a pharmaceutical composition thereof to an individual in need thereof.

In another aspect, the present invention also provides methods for preventing and/or treating AXL kinase-mediated diseases or disease states, it comprises administering the crystal form B of the present invention or a pharmaceutical composition thereof to an individual in need thereof.

In another aspect, the present invention also provides methods for preventing and/or treating AXL kinase-mediated diseases or disease states, it comprises administering the crystal form C of the present invention or a pharmaceutical composition thereof to an individual in need thereof.

In another aspect, the present invention also provides mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof for use in the prevention and/or treatment of AXL kinase-mediated diseases or disease states.

In another aspect, the present invention also provides the compound of formula I of the present invention for use in the prevention and/or treatment of AXL kinase-mediated diseases or disease states.

In another aspect, the present invention also provides the compound of formula II of the present invention for use in the prevention and/or treatment of AXL kinase-mediated diseases or disease states.

In another aspect, the present invention also provides the compound of formula III of the present invention for use in the prevention and/or treatment of AXL kinase-mediated diseases or disease states.

In another aspect, the present invention also provides the crystal form composition of the present invention for use in the prevention and/or treatment of AXL kinase-mediated diseases or disease states.

In another aspect, the present invention also provides the crystal form A of the present invention or pharmaceutical composition thereof for use in the prevention and/or treatment of AXL kinase-mediated diseases or disease states.

In another aspect, the present invention also provides the crystal form B of the present invention or pharmaceutical composition thereof for use in the prevention and/or treatment of AXL kinase-mediated diseases or disease states.

In another aspect, the present invention also provides the crystal form C of the present invention or pharmaceutical composition thereof for use in the prevention and/or treatment of AXL kinase-mediated diseases or disease states.

In some embodiments, the AXL kinase-mediated disease or disease state is cancer.

In some typical embodiments, the cancers are associated with hematologic neoplasms.

The mono-p-toluenesulfonate of the compound of formula IV prepared by the present invention, hydrate thereof and the crystal form of the hydrate have good stability, solving the problem of the instability of the free base (compound of formula IV) at high temperature, high humidity, and under the conditions of illumination, and the crystal form obtained by the preparation method has the advantages of being stable, easy to be processed, and has high solubility.

### Relevant Definitions

Unless otherwise specified, the following terms used in the description and claims have the following meanings:

The term “pharmaceutically acceptable carrier” refers to those carriers that have no obvious irritating effect on the body and do not impair the biological activity and performance of the active compound. Including but not limited to any diluent, disintegrant, binder, glidant, and wetting agent approved by the National Medical Products Administration for use on humans or animals.

The “X-ray powder diffraction pattern spectrum” in the present invention is obtained by using Cu-Kα radiation measurement.

“2θ” or “2θ angle” in the present invention refers to the diffraction angle, θ is the Bragg angle, and the unit is ° or degree; the error range of each characteristic peak 2θ is ±0.20°.

It should be noted that in X-ray powder diffraction spectroscopy (XRPD), the diffraction spectra obtained from crystalline compounds tend to be characteristic for a particular crystallization, where the relative intensities of the bands (especially at low angles) may vary due to the dominant orientation effect resulting from differences in crystallization conditions, particle size, and other measurement conditions. Therefore, the relative intensities of the diffraction peaks are not characteristic of the crystallization in question. It is the relative positions of the peaks rather than their relative intensities that should be taken into account when determining whether the crystallization is the same as a known one. In addition, it is well known in the field of crystallography that there may be a slight error in the position of the peaks for any given crystallization. For example, the position of the peaks can shift due to changes in temperature when analyzing the sample, sample movement, or instrument calibration, and the error in the determination of the 2θ value is sometimes about ±0.2°. Therefore, this error should be taken into account when determining each crystal structure. In the XRPD spectrum, the 2θ angle or crystal plane distance d is usually used to indicate the peak position, and the two have a simple conversion relationship: d=λ/2 sin θ, wherein d represents the crystal plane distance, λ represents the wavelength of the incident X-rays, and θ is the diffraction angle. For the same crystallization of the same compound, the peak positions of its XRPD spectra have similarity in the whole, and the relative intensity error may be larger. It should also be noted that in the identification of mixtures, some diffraction lines are missing due to factors such as decreasing content, etc. In this case, it is not necessary to rely on all the spectral bands observed in the high-purity specimen, and even a single band may be characteristic for a given crystallization.

Differential Scanning Calorimetry (DSC) determines the transition temperature when a crystal absorbs or releases heat as a result of changes in its crystal structure or as a result of crystal melting. For the same crystalline form of the same compound, the thermal transition temperature and melting point are typically within about 5° C. of each other in successive analyses, and usually within about 3° C. of each other. When describing a compound as having a given DSC peak or melting point, this refers to the DSC peak or melting point 5° C. DSC provides an aid in recognizing different crystal forms. Different crystal forms can be recognized based on their different transition temperature characteristics. It should be noted that for mixtures, the DSC peak or melting point may fluctuate over a wider range. In addition, the melting temperature is related to the rate of heating, since decomposition occurs during the melting process.

Thermogravimetric analysis (TGA) refers to a thermal analysis technique that measures the relationship between the mass of a sample to be measured and the change in temperature at a programmed temperature. When the substance to be measured is sublimated or vaporized during the heating process, resulting in the decomposition into gas or the loss of crystalline water of crystallization, causing a change in the mass of the substance to be measured. In this case, the thermogravimetric curve is not straight but decreases. By analyzing the thermogravimetric curve, it is possible to know at what temperature the substance to be measured changes, and according to the weight lost, it is possible to calculate the amount of substance lost.

When referring to, for example, an XRPD pattern, a DSC pattern or a TGA pattern, the term “as shown” includes patterns that are not necessarily the same as those depicted herein, but which fall within the limits of experimental error when considered by one skilled in the art.

As used herein, the term “hydrate” is a specific solvent compound in which the solvent is water, and examples of hydrates include hemihydrate, monohydrate, sesquihydrate, dihydrate, and the like.

Different crystalline forms of a particular substance, such as a salt of the present invention, may include both an anhydrous form of the substance and a hydrated form of the substance, wherein each of the anhydrous form and the hydrated form are distinguishable from each other by a different XRPD image and thus by representing a different crystal lattice. In some examples, a single crystalline form (e.g., identified by a separate XRPD image) may have a variable water content or solvent content, wherein the lattice remains essentially unchanged (as represented by the XRPD image) except for changes in composition relative to water and/or solvent.

Unless otherwise specified, the abbreviations of the present invention have the following meanings:
M: mol/L
mM: mmol/L
nM: nmol/L
Boc: tert-butoxycarbonyl
¹HNMR: Hydrogen Nuclear Magnetic Resonance Spectrum
MS(ESI+): Mass Spectrometry
DMSO-d₆: deuterated dimethyl sulfoxide
CDCl₃: deuterated chloroform
DTT: dithiothreitol
SEB: SupplementedEnzymaticBuffer (supplemented enzyme buffer)
IMDM (Iscove's Modified Dulbecco's Medium): Iscove's modified Dulbecco's medium.
Room temperature: 25° C.

### BRIEF DESCRIPTION OF THE DRAWINGS

In order to more clearly illustrate the technical solutions of the embodiments and prior art of the present invention, the following is a brief introduction to the embodiments and the prior art need to use the accompanying drawings, it is obvious that the following description of the accompanying drawings is only some of the embodiments of the present invention, for the person of ordinary skill in the field, according to these drawings, there are also other attached drawings can also be obtained.

FIG. 1 is the X-ray powder diffraction (XRPD) spectrum of the crystal form B in Example 3.

FIG. 2 is a differential scanning calorimetry (DSC) spectrum of the crystal form B in Example 3.

FIG. 3 is the thermogravimetric (TGA) spectrum of the crystal form B in Example 3.

FIG. 4 is the FT-IR (FT-IR) spectrum of the crystal form B in Example 3.

FIG. 5 is the FT-Raman spectrum of the crystal form B in Example 3.

FIG. 6 is the dynamic moisture adsorption (DVS) spectrum of the crystal form B in Example 3.

FIG. 7 is the X-ray powder diffraction (XRPD) spectrum of the crystal form C in Example 6.

FIG. 8 is the X-ray powder diffraction (XRPD) spectrum of the crystal form A in Example 8.

FIG. 9 is the X-ray powder diffraction (XRPD) spectrum of the mesylate salt of the compound IV in Example 10.

FIG. 10 is the X-ray powder diffraction (XRPD) spectrum of the monohydrochloride of the compound of formula IV in Example 10.

FIG. 11 is the X-ray powder diffraction (XRPD) spectrum of the dihydrochloride of the compound of formula IV in Example 10.

FIG. 12 is the X-ray powder diffraction (XRPD) spectrum of the phosphate of the compound of formula IV in Example 10.

FIG. 13 is the X-ray powder diffraction (XRPD) spectrum of the hippurate of the compound of formula IV in Example 10.

FIG. 14 is the X-ray powder diffraction (XRPD) spectrum of the sulfate of the compound of formula IV in Example 10.

FIG. 15 is the X-ray powder diffraction (XRPD) spectrum of the hydrobromide salt of the compound of formula IV in Example 10.

FIG. 16 is the X-ray powder diffraction (XRPD) spectrum of the benzene sulfonate salt of the compound of formula IV in Example 10.

FIG. 17 is the X-ray powder diffraction (XRPD) spectrum of the oxalate salt of the compound of formula IV in Example 10.

FIG. 18 is the X-ray powder diffraction (XRPD) spectrum of the fumarate salt of the compound of formula IV in Example 10.

FIG. 19 is the X-ray powder diffraction (XRPD) spectrum of the citrate salt of the compound of formula IV in Example 10.

FIG. 20 is an X-ray powder diffraction (XRPD) spectrum of the compound of formula I prepared in Example 11.

### DETAILED DESCRIPTION

The present invention is described in more detail below by means of examples. However, these specific descriptions are only used to illustrate the technical solutions of the present invention and do not constitute any limitation on the present invention.

The test conditions for each instrument are as follows:

### (1) X-Ray Powder Diffraction (XRPD)

Instrument model: Bruker D2 Phaser2^{nd}
Reflective Mode

| X-ray | Cu, kα, Kα1(Å): 1.540598, |
|---|---|
| | Kα2(Å): 1.544426 |
| | Kα2/Kα1 intensity ratio: 0.50 |
| X-ray tube settings | 30 kV, 10 mA |
| Detector | 1D-LYNXEYE |
| Sola slit | 0.6 mm |
| Sample rotation time (seconds) | 15 |
| Scan range (°2θ) | 3-40° |
| Scan step size (°2θ) | 0.043 |
| Scan time (seconds) | 150 |

### (2) Thermogravimetric Analyzer (TGA)

Instrument model: TA Instruments TGA 25

Purge gas: nitrogen

Heating rate: 10° C./min

Heating range: room temperature −300° C.

Method: placed the sample on an aluminum plate, then placed the aluminum plate on a platinum plate, and heated it from room temperature to the set temperature at a speed of 10° C./min in an open nitrogen atmosphere.

### (3) Differential Scanning Calorimeter (DSC)

Instrument model: TA Instruments DSC 25

Purge gas: nitrogen.

Heating rate: 10° C./min

Heating range: 20-300° C.

Method: placed the sample on an aluminum plate, capped and heated the sample from 20° C. to the set temperature under nitrogen atmosphere at a rate of 10° C./min.

### (4) Fourier Transform Infrared Spectroscopy (FT-IR)

Instrument model: Thermo Fourier transform infrared spectrometer ID1-summit

Instrument calibration: polystyrene film

Test conditions: KBr tableting method

### (5) Fourier Transform Raman Spectroscopy (FT-Raman)

Instrument model: Nicoret Fourier transform Raman spectrometer DXR780

Exposure time: 20 s

Number of exposures: 10 times

Light source: 780 nm

Slit: 400 lines/mm

Laser intensity: 14 mW

Scanning range: 50 cm-1-3000 cm-1

### (6) Dynamic Vapor Sorption (DVS)

Instrument model: Surface Measurement System(SMS)-DVS Intrinsic

DVS

| Temperature | 25° | C. |
|---|---|---|
| Sample quantity | 10-20 | mg |
| Protective gas and flow rate | N₂, 200 mL/min | |
| dm/dt | 0.002%/min | |
| Minimum dm/dt balancing time | 10 | min |
| Maximum balancing time | 180 | min |

| | |
|---|---|
| Humidity range | Indoor humidity-95% |
| | RH-0% RH-95% RH |
| Humidity gradient | 10%(90% RH-0% RH-90% RH) |

The specific instrument setting parameters are as follows:

### (7) Single Crystal Diffractometer and its Parameter Information:

Single Crystal X-ray Diffraction (SCXRD)

Instrument model: Bruker D8 Venture

Light source: Cu-Kα, λ=1.54 Å

Detector: CMOS area detector

Resolution: 0.8 Å

X-ray tube settings: Tube voltage 50 KV, tube current 1.2 mA

Exposure time: 50 s

Test temperature: 170(2)K

### (8) Analysis Conditions of HPLC

| Equipment | High performance liquid chromatography | | |
|---|---|---|---|
| | (PDA detector) | | |
| Column | TitankC18 (4.6 mm × 150 mm, 3 μm) or a | | |
| | chromatography column of equivalent performance | | |
| Wavelength | 280 nm | Flow | 1.0 |
| (nm) | | rate(ml/min) | |
| Column | 30 | Detection | 60 |
| temperature | | time (min) | |
| (° C.) | | | |
| Injection | 10 | Elution | gradient |
| volume (μL) | | method | elution |
| Mobile phase | A | 0.01 mol/L potassium dihydrogen | |
| | | phosphate buffer (1.36 g potassium | |
| | | dihydrogen phosphate was dissolved | |
| | | in 1 L of water and pH was adjusted | |
| | | to 7.0 with potassium hydroxide solution) | |
| | B | Acetonitrile | |
| | Time | Mobile | Mobile |
| Run method | (min) | phase A (%) | phase B (%) |
| | 0 | 85 | 15 |
| | 10 | 70 | 30 |
| | 15 | 70 | 30 |
| | 25 | 20 | 80 |
| | 30 | 20 | 80 |
| | 31 | 85 | 15 |
| | 40 | 85 | 15 |

### Example 1 Preparation of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide

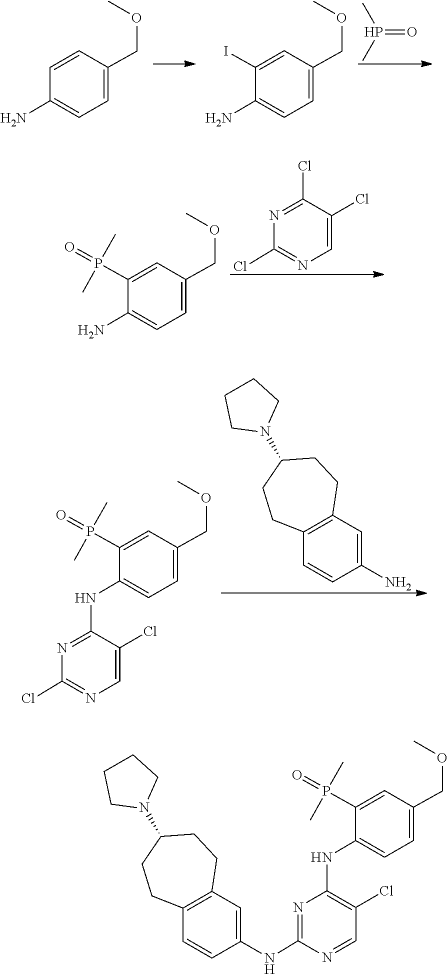

### a) 2-Iodo-4-(methoxymethyl)aniline

4-(Methoxymethyl)aniline (9 g), iodine (16.65 g) and sodium bicarbonate (16.53 g) were added to a solution of dichloromethane (261 mL)/water (135 mL), and the reaction solution was stirred at 22° C. for 16 h. The reaction solution was quenched with saturated sodium thiosulfate (10 mL) at room temperature. The resulting mixture was extracted with dichloromethane (3×100 mL), then the combined organic layers were washed with saturated aqueous sodium chloride solution (1×100 mL) and dried over anhydrous sodium sulfate. After filtration, the filtrate was concentrated under reduced pressure. The residue was purified by silica gel column chromatography (petroleum ether/ethyl acetate=1/1, v/v) to obtain the title product (16 g). MS(ESI+): 264.0 (M+H).

### b) (2-Amino-5-(methoxymethyl)phenyl)dimethyl phosphine oxide

To a stirred solution of 2-iodo-4-(methoxymethyl)aniline (16 g, 60.82 mmol, 1.00 eq.), potassium phosphate (14.20 g), palladium acetate (0.68 g), and 4,5-bis(diphenylphosphino)-9,9-dimethyloxanthene (1.76 g) in N,N-dimethylformamide (224 mL) under a nitrogen atmosphere was added dimethyl phosphine oxide (5.22 g), and the reaction was stirred at 120° C. for 2 hours. The mixture was cooled to room temperature. The resulting mixture was filtered and the filter cake was washed with N, N-dimethylformamide (3×5 mL). The filtrate was concentrated under reduced pressure and purified by silica gel column chromatography (dichloromethane/methanol=20/1, v/v) to give the title product (12.9 g). MS(ESI+): 214.1 (M+H).

### c) (2-((2,5-Dichloropyrimidin-4-yl)amino)-5-(methoxymethyl)phenyl)dimethyl phosphine oxide

(2-Amino-5-(methoxymethyl)phenyl)dimethyl phosphine oxide (1.10 g), 2,4,5-trichloropyrimidine (1.23 g) and N,N-diisopropylethylamine (2.00 g) were added to N,N-dimethylformamide (22 mL) at room temperature and stirred for 3 h. The resulting mixture was diluted with dichloromethane (30 mL). The reaction was quenched by adding water (10 mL) at 0° C. The resulting mixture was extracted with dichloromethane (3×50 mL). The combined organic layers were washed with saturated sodium chloride (1×50 mL) and dried over anhydrous sodium sulfate. After filtration, the filtrate was concentrated under reduced pressure. Purification of the residue by silica gel column chromatography (dichloromethane/methanol=20/1, v/v) gave the title product (1.28 g). MS(ESI+): 360.0 (M+H).

### d) (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide

(2-((2,5-Dichloropyrimidin-4-yl)amino)-5-(methoxymethyl)phenyl)dimethylphosphine oxide (50.00 mg) and (S)-7-(pyrrolidin-1-yl)-6,7,8,9-tetrahydro-5H-benzo[7]annulen-2-amine (31.98 mg) were added to isopropanol (2 mL), followed by hydrogen chloride in 1,4-dioxane (10 drops, 4M) and microwave radiation was applied at 130° C. for 3.5 h. The mixture was then cooled to room temperature and concentrated under reduced pressure. The crude product was purified by reversed-phase high performance liquid chromatography (column YMCActusTriartC18, 30*150 mm, particle size 5 μm, mobile phase A: water (10 mmol/L ammonium bicarbonate), mobile phase B: acetonitrile, flow rate: 60 mL/min, gradient: 20% B to 50% B, 8 min, wavelength: 220 nm, retention time: 6.83 min, column temperature: 25° C.), the title product (20.2 mg) was obtained.

¹HNMR (400 MHz, DMSO-d₆,ppm)

δ11.07 (s, 1H), 9.26 (s, 1H), 8.52 (d, J=4.6 Hz, 1H), 8.17 (s, 1H), 7.53 (dd, J=14.0, 2.0 Hz, 1H), 7.44 (q, J=3.1 Hz, 2H), 7.26 (dd, J=8.1, 2.3 Hz, 1H), 6.97 (d, J=8.1 Hz, 1H), 4.42 (s, 2H), 3.31 (s, 3H), 3.01-2.75 (m, 2H), 2.55 (s, 5H), 2.50 (s, 2H), 1.84 (s, 2H), 1.81 (s, 3H), 1.77 (s, 3H), 1.70 (q, J=3.6, 3.2 Hz, 4H), 1.54 (s, 2H). MS(ESI+): 554.2 (M+H).

### Example 2 Activity Assay

Related compounds prepared in Example 1 were tested for enzyme activity, cell activity, and activity in vivo.

The specific structure of the positive drug 1 (BGB324) used in the activity test is as follows:

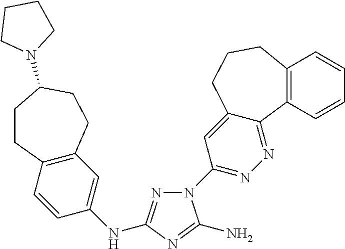 [SMILES: Nc1nc(Nc2ccc3c(c2)CC[C@@H](N2CCCC2)CC3)nn1-c1cc2c(nn1)-c1ccccc1CCC2]

The specific structure of positive drug 2 (TP0903) is as follows:

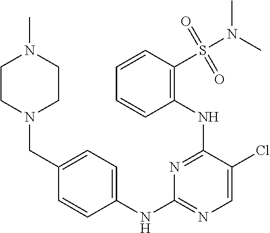 [SMILES: CN1CCN(Cc2ccc(Nc3ncc(Cl)c(Nc4ccccc4S(=O)(=O)N(C)C)n3)cc2)CC1]

The above compounds were purchased from Shanghai Shenghong Biotechnology Co., Ltd.

### (1) AXL Kinase Inhibitory Activity

### 1. Experimental Process

a) AXL enzyme (Carna, 08-107) configuration and addition: 33.33 ng/μL of AXL enzyme was diluted to 0.027 ng/μL (1.67×, final conc.=0.016 ng/μL) with 1× enzyme buffer (1 ml of 1× enzyme buffer configured with 200 μL of Enzymatic buffer kinase 5×, 10 μL of 500 mM MgCl₂, 10 μL of 100 mM DTT, and 6.26 μL of 2500 nM SEB, with the addition of 773.75 μL of H₂O), using a BioTek (MultiFloFX) automated dispenser, compound wells and positive control wells were each spiked with 6 μL of 1.67 times the final concentration of enzyme solution; 6 μL of 1× Enzymatic buffer was added to the negative control wells.

b) Compound preparation and addition: the compounds prepared in Example 1 and the positive drug were diluted from 10 mM to 100 μM using DMSO and titrated with a compound titrator (Tecan, D300e), which automatically sprayed the desired concentration into each well, with a 1st concentration of 1 μM and ½ log gradient dilution, for a total of 8 concentrations. Centrifugation was performed at 2500 rpm for 30 s, and incubation was performed at room temperature 15 minutes.

c) ATP, substrate preparation and addition: ATP (Sigma, A7699) was diluted using 1× enzyme buffer, from 10 mM to 75 μM (5×), resulting in a final concentration of 15 μM; The substrate TKSubstrate 3-biotin (Cisbio, 61TKOBLC) was diluted with 1× enzyme buffer, from 500 μM to 5 μM (5×), resulting in a final concentration of 1 μM; ATP was mixed with the substrate in equal volumes. Using the BioTek automatic liquid dispenser, 4 μL of the mixture was added to each well; The plate was centrifuged at 2500 rpm for 30 seconds, followed by a 45-minute reaction at 25° C.

d) Assay preparation and addition: Streptavidin-XL665 (Cisbio, 610SAXLG) was diluted with HTRFK in EASE detection buffer (cisbio) from 16.67 μM to 250 nM (4×) with a final concentration of 62.5 nM; TK Antibody-Cryptate (Cisbio) was diluted from 100× to 5× with HTRF KinEASE detection buffer (cisbio), and the final concentration was 1×; XL665 was mixed in equal volume with Antibody, and 10 μL was added to each well with a BioTek automated dispenser, centrifugation was performed at 2500 rpm for 30 s, and the reaction was carried out at 25° C. for 1 hour. At the end of the reaction, the reaction was detected by a multifunctional plate reader HTRF.

### 2. Data Analysis

The IC₅₀ values of compounds for AXL kinase inhibition were obtained by fitting the dose-response curves using GraphPad Prism 5 software log(inhibitor) vs. response-Variable slope.

The inhibition rate calculation formula is as follows:

$$Inhibition rate \% = \frac{\begin{matrix}Conversion readings for wells without compounds inhibition - \\ conversion readings for samples\end{matrix}}{\begin{matrix}Conversion readings for wells without compounds inhibition - \\ conversion readings for wells without enzyme activity\end{matrix}} \times 100 \%$$

### 3. The Experimental Results are Detailed in the Following Table

TABLE 6
IC₅₀ data for AXL inhibitory activity of Example 1 compounds

| Compound | AXL inhibitory activity IC₅₀ (nM) |
|---|---|
| Example 1 | 4.89 |
| Positive drug 1 (BGB324) | 2.25 |
| Positive drug 2 (TP0903) | 16.39 |

### (2) Detection of Cell Proliferation Inhibition by Compounds

### 1. Experimental Process

MV-4-11 cells (human myelomonocytic leukemia cell line, medium: IMDM+10% fetal bovine serum) were purchased from Nanjing Kobai Biotechnology Co. Ltd. and were cultured in an incubator at 37° C., 5% CO₂. The cells in logarithmic growth phase were spread in 96-well plates at a cell density of 8000 cells/well, 6000 cells/well, 5000 cells/well, 4000 cells/well and 3000 cells/well, respectively, and a blank control group was set up at the same time.

The compounds to be tested as well as the positive drug were dissolved in dimethyl sulfoxide to prepare a 10 mM reservoir solution, which was stored at −80° C. in a refrigerator for a long period of time. After 24 hours of cell spreading, the 10 mM compound reservoir solution was diluted with dimethyl sulfoxide to obtain a 200-fold concentration of working solution (ranging from 200 to 2000 μM, with a 3-fold gradient, and a total of 10 concentrations). 3 μL of each concentration was added to 197 μL of complete medium to further dilute it to a 3-fold concentration of working solution. Subsequently, 50 μL of this working solution was added to 100 μL of cell culture medium (with a final dimethyl sulfoxide concentration of 0.5%, v/v), with two replicate wells per concentration. After 72 hours of dosing treatment, 50 μL of Cell Titer-Glo® (purchased from Promega) was added to each well.

Fluorescence signals were measured on an Envision plate reader (PerkinElmer) following the procedure in the instruction manual, and the IC₅₀ value of the compound on cell proliferation inhibition was obtained by fitting the dose-response curve using the GraphPad Prism 5 software log(inhibitor) vs. response-variable slope.

Inhibition rate calculation formula:

$$Inhibition rate \% = \frac{1 - ( Subject signal value - Blank group signal value )}{Negative control group signal value - Blank group signal value} \times 100 \%$$

Wherein:
Subject signal value: mean fluorescence signal value of cells+culture medium+compound group.
Blank group signal value: mean fluorescence signal value of culture medium group (containing 0.5% DMSO).
Negative control group signal value: mean fluorescence signal of cell+culture medium group (containing 0.5% DMSO).

### 2. Experimental Results

The IC₅₀ (MV4-11, nM) of the anti-proliferative activity of the compound of Example 1 on MV4-11 cells is 6.97.

### (3) In Vivo Efficacy of MV4-11 of the Compound

Inhibitory effects of test compounds as well as positive drugs on in vivo tumor growth in a transplanted tumor model of human acute monocytic leukemia cells MV-4-11 in nude mice.

### 1. Construction of Mouse Model

The logarithmic growth phase MV-4-11 cells were harvested, the cells were counted and resuspended, and then the cell concentration was adjusted to 7.0×10⁷ cells/mL; the MV-4-11 cells were injected subcutaneously into the anterior right axilla of nude mice, and each animal was inoculated with 200 μL (14×10⁶ cells/animal), to establish the MV-4-11 transplantation tumor model. When the tumor volume reached 100-300 mm³, the tumor-bearing mice with good health condition and similar tumor volume were selected.

### 2. Configuration of Compounds

The compound as well as the positive drug, were vortexed and shaken with an appropriate solvent and then sonicated to dissolve completely and then the appropriate volume of citrate buffer was slowly added and vortexed and shaken to mix the liquids well to obtain the administered formulations at concentrations of 0.1, 0.5, and 1 mg·mL⁻¹.

Solvent control group: PEG400&citric acid buffer (20:80, v:v).

### 3. Animal Grouping and Drug Administration

The modeled mice were randomly divided into groups (n=6). On the day of grouping, the relevant compounds and positive drugs were administered. The experiment was concluded after 21 days or when the tumor volume reached 2000 mm³ in the solvent control group (whichever occurred first). The administration volume was 10 mL/kg. Both the compounds and the positive drugs were administered intragastrically once a day. Throughout the experiment, tumor dimensions and animal weights were measured twice a week to calculate tumor volume.

### 4. Data Analysis

Tumor volume (TV) was calculated as: tumor volume (mm³)=l×w²/2,

wherein, l represents the long diameter of the tumor (mm); w represents the short diameter of the tumor (mm).

Relative Tumor Volume (RTV) was calculated as: RTV=TVₜ/TVᵢₙᵢₜᵢₐₗ, wherein, TVᵢₙᵢₜᵢₐₗ is the tumor volume measured during group administration; TVₜ is the tumor volume measured at each time during the administration period.

The tumor growth inhibition rate TGI (%) was calculated as: TGI=100%×[1−(TV_{t(T)}−TV_{initial(T)})/(TV_{t(C)}-TV_{initial(C)}],

wherein, TV_{t(T)} represents the tumor volume per measurement in the treatment group; TV_{initial(T)} represents the tumor volume in the treatment group at the time of group administration; TV_{t(C)} represents the tumor volume per measurement in the solvent control group; and TV_{initial(C)} represents the tumor volume in the solvent control group at the time of group administration.

The relative tumor proliferation rate (% T/C) was calculated as: % T/C=100%×(RTV_{T}/RTV_{C}),
wherein, RTV_{T} represents the RTV of the treatment group; RTV_{C} represents the RTV of the solvent control group.

The experimental data were calculated and related statistical were processed using Microsoft Office Excel 2007 software.

### 5. The Experimental Results are as Follows

TABLE 7
In vivo efficacy of compounds of Example 1

| | intragastrical | Tumor growth | Relative tumor |
|---|---|---|---|
| | administration | inhibition | proliferation |
| Compound | dosage (mg/kg/d) | rate (TGI %) | rate (% T/C) |
| Example 1 | 5 | 85 | 23 |
| Positive drug 1 | 20 | 32 | 71 |
| (BGB324) | | | |
| Positive drug 2 | 5 | 62 | 42 |
| (TP0903) | | | |

Note:
The experimental data in the table are the relevant data obtained when the experiment ends (the end of the experiment is defined as: 21 days later or when the tumor volume in the solvent control group reaches 2000 mm³ (whichever reaches the indicator first)).

### (4) Pharmacokinetic Study of Compounds in ICR Mice

### 1. Gavage Prescription Configuration of Compounds

Each compound was prepared into a 10 mg/mL stock solution with DMSO.

Mixed solvent preparation: Tween 80:PEG 400:Water=1:9:90 (v/v/v).

Accurately aspirated 450 μL of DMSO stock solution of the compounds at a concentration of 10 mg/mL to a glass vial, respectively, added an appropriate volume of DMSO and mixed solvent, the ratio of solvent in the final preparation is DMSO:mixed solvent (v/v)=10:90, vortexed (or sonicated), dispersed homogeneously, to obtain the concentration of 1 mg/mL of 4.5 mL of the administered test solution respectively.

### 2. Test Plan

Male 6-10 weeks old ICR mice (mouse source: Viton Lever Laboratory Animal Technology Co., Ltd.) were taken, 6 mice in each group, and the mice were fasted overnight and fed 4 hours after drug administration. On the day of the experiment, mice were given 10 mg·kg⁻¹ of compound test solution by gavage respectively. After the administration of the drug, mice were blood sampled at 0, 5 min, 15 min, 30 min, 1 h, 2 h, 4 h, 8 h, and 24 h from the orbital region of about 100 μp, which was placed in EDTA-K₂ anticoagulant tubes. The whole blood samples were centrifuged at 1500-1600 g for 10 min, and the plasma obtained from the separation was stored in a refrigerator at −40˜−20° C. and used for the analysis of biological samples. Plasma concentration was determined by LC-MS/MS method.

### 3. Data Analysis and Results

Pharmacokinetic parameters were calculated using the non-compartment model in Pharsight Phoenix 7.0, and the results are detailed in the following table.

TABLE 8
Mouse pharmacokinetic results of the compound of Example 1

| Compound | Cmax(ng/ml) | Tmax(h) | AUC₀₋₂₄(ng · h/mL) | T_{1/2}(h) |
|---|---|---|---|---|
| Example 1 | 324 | 2.17 | 1300 | 1.35 |
| Positive drug 2 | 26.8 | 0.25 | 52.2 | 1.20 |
| (TP-0903) | | | | |

### Example 3 Preparation of Crystal Form B of Mono-p-Toluenesulfonate Monohydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (Compound of Formula II)

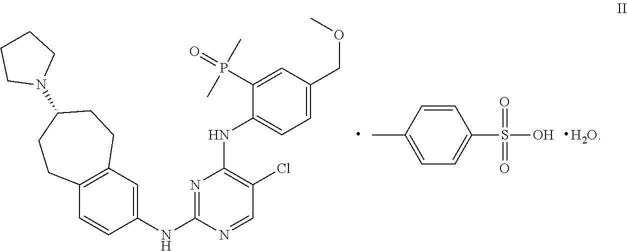

P-toluenesulfonic acid monohydrate (6.87 g) and 99% isopropanol-water (volume percentage, 140 mL) were added to a 500-mL double glass jacketed reactor and the mixture was heated to 70° C. and stirred mechanically to dissolve. (S)-(2-((5-chloro-2-((7-(pyrrolidin-1-yl)-6,7,8,9-tetrahydro-5H-benzo[7]annulen-2-yl)amino))pyrimidin-4-yl)amino)-5-(methoxymethyl)phenyl)dimethyl phosphine oxide (compound of formula IV, prepared according to the method of Example 1) (20 g) was dissolved in 99% isopropanol-water (volume percentage, 60 mL) and added dropwise to the reactor for about 15 minutes. After stirring for a few minutes, a large amount of solid was precipitated in the reactor and stirring was continued for about 15 minutes. The system was cooled to 60° C., and methyl tert-butyl ether (200 mL) was added to the reactor. After the addition, the system was stirred and ripened at 60° C. for 1 hour. After ripening, the system was cooled to 20° C., and the stirring and ripening continued for another hour. After ripening, the system was filtered and the wet cake was dried under vacuum at 40° C. for 15 hours to obtain light yellow solid powdery crystal form B of mono-p-toluenesulfonate monohydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide: 23.67 g (yield: 88.3%).

¹HNMR (400 MHz, DMSO-d6): 11.11 (s, 1H); 9.40 (br, 1H); 9.37 (s, 1H); 8.56-8.54 (m, 1H); 8.19 (s, 1H); 7.56 (dd, J=14.2 Hz, 1H); 7.50-7.45 (m, 4H); 7.37 (dd, J=8.2, 2.2 Hz, 1H); 7.11 (d, J=7.9, 2H); 7.04 (d, J=8.2, 1H); 4.44 (s, 2H); 3.48 (s, 3H); 3.33 (s, 3H); 3.15 (s, 2H); 2.82-2.62 (m, 4H); 2.32 (s, 2H); 2.29 (s, 3H); 1.98 (s, 2H); 1.85-1.83 (m, 2H); 1.81 (d, J=2.4 Hz, 3H); 1.77 (d, J=2.4 Hz, 3H); 1.39 (q, J=11.9 Hz, 2H).

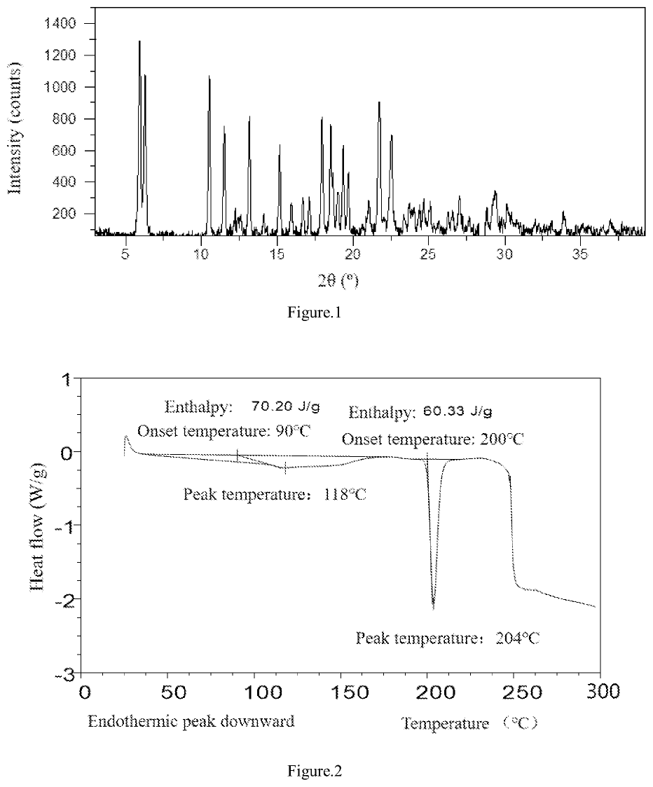

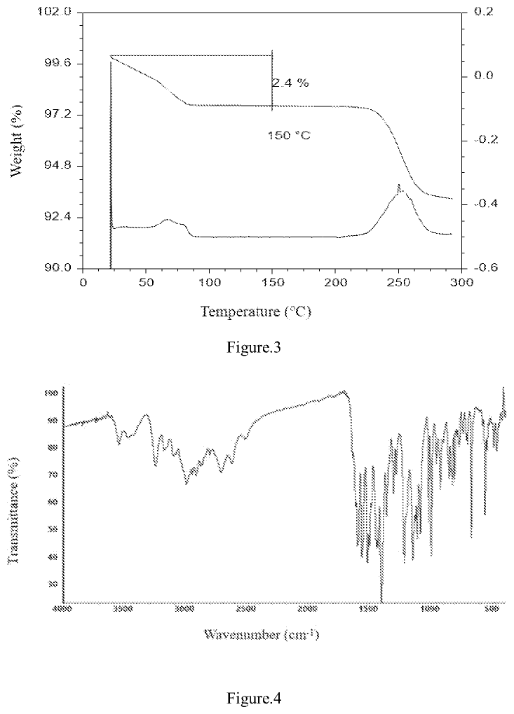

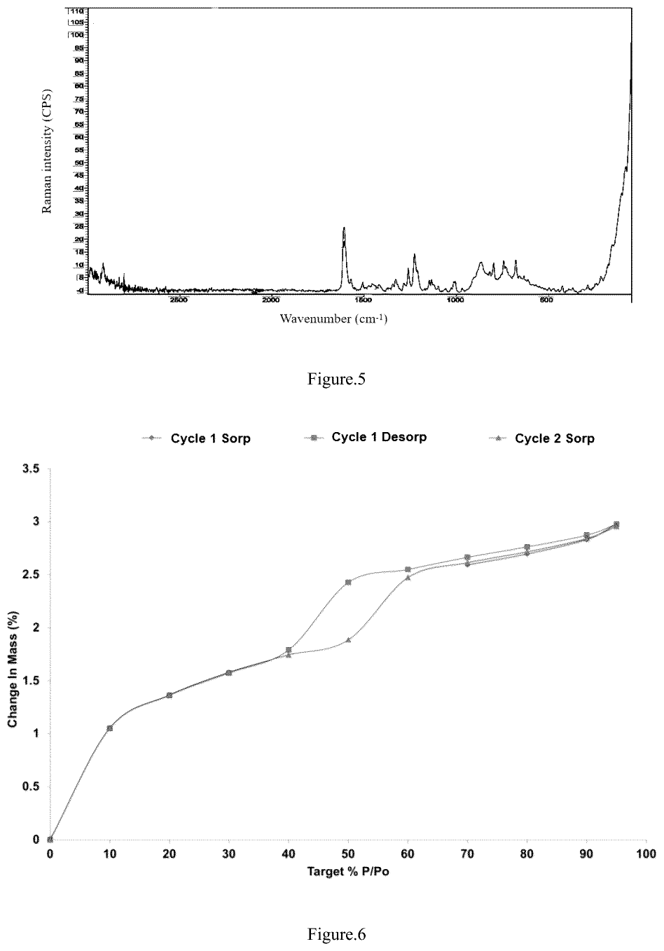

The X-ray powder diffraction pattern data is shown in Table 9 and the spectrum is shown in FIG. 1; the DSC spectrum shows endothermic peaks at 90° C. and 200° C., see FIG. 2 for details; the TGA spectrum shows a weight loss of 2.4% at 25-150° C., see FIG. 3 for specific data; see FIG. 4 for detailed FT-IR spectrum, FIG. 5 for detailed FT-Raman spectrum, and FIG. 6 for detailed DVS spectrum.

TABLE 9
X-ray powder diffraction pattern data of crystal form B

| 2θ(°) | Counts | I/I₀(%) |
|---|---|---|
| 6.0 | 1772 | 100.0 |
| 6.3 | 1424 | 80.3 |
| 7.7 | 36 | 2.0 |
| 8.1 | 26 | 1.4 |
| 9.2 | 12 | 0.7 |
| 9.7 | 16 | 0.9 |
| 10.5 | 1448 | 81.7 |
| 11.5 | 970 | 54.8 |
| 12.3 | 215 | 12.1 |
| 12.4 | 133 | 7.5 |
| 12.6 | 169 | 9.5 |
| 13.2 | 1033 | 58.3 |
| 13.6 | 53 | 3.0 |
| 14.1 | 190 | 10.7 |
| 15.2 | 804 | 45.4 |
| 15.9 | 281 | 15.9 |
| 16.7 | 323 | 18.2 |
| 17.1 | 292 | 16.5 |
| 18.0 | 974 | 54.9 |
| 18.6 | 947 | 53.4 |
| 18.7 | 449 | 25.3 |
| 19.0 | 354 | 20.0 |
| 19.4 | 764 | 43.1 |
| 19.7 | 487 | 27.5 |
| 20.6 | 50 | 2.8 |
| 21.1 | 239 | 13.5 |
| 21.8 | 1152 | 65.0 |
| 22.6 | 844 | 47.6 |
| 23.4 | 106 | 6.0 |
| 23.7 | 232 | 13.1 |
| 24.1 | 184 | 10.4 |
| 24.4 | 181 | 10.2 |
| 24.7 | 274 | 15.5 |
| 25.1 | 227 | 12.8 |
| 25.6 | 71 | 4.0 |
| 26.3 | 177 | 10.0 |
| 26.6 | 223 | 12.6 |
| 27.0 | 371 | 20.9 |
| 27.7 | 249 | 14.1 |
| 28.0 | 143 | 8.1 |
| 28.3 | 226 | 12.8 |
| 28.7 | 109 | 6.2 |
| 28.8 | 303 | 17.1 |
| 29.4 | 459 | 25.9 |
| 30.2 | 281 | 15.8 |
| 32.0 | 81 | 4.5 |
| 33.1 | 68 | 3.9 |
| 33.9 | 152 | 8.6 |
| 35.1 | 58 | 3.3 |
| 35.7 | 44 | 2.5 |

### Example 4 Preparation of Crystal Form B of Mono-p-Toluenesulfonate Monohydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (Compound of Formula II)

(S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (compound of formula IV, 2.34 g), p-toluenesulfonic acid monohydrate (0.80 g) and ethanol (11.7 mL) were added to a 50-mL glass bottle and the mixture was dissolved with magnetic stirring at room temperature. A large amount of solid was precipitated after about 10 minutes. Continued stirring for about 30 minutes. Then, isopropyl acetate (11.7 mL) was added to the glass bottle. After addition, the mixture was stirred and ripened for about 30 minutes at room temperature. After ripening, isopropyl acetate (11.7 mL) was added to the glass bottle and continued ripening for about 1.5 hours. After ripening, filtration was carried out and the wet filter cake was dried under vacuum at 40° C. for 24 hours to obtain light yellow solid powdery crystal form B of mono-p-toluenesulfonate monohydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide: 2.35 g (yield: 74.8%).

### Example 5 Preparation of Single Crystal of Crystal Form B of Mono-p-Toluenesulfonate Monohydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (Compound of Formula II)

Single crystal culture method: the sample of monohydrate crystal form B was dissolved in 95% ethanol-water, prepared as a suspension of 9.1 mg/mL, warmed up to 50-60° C. to dissolve, filtered and precipitated by cooling down at room temperature that is to obtain the elongated needle-like single crystals, and its crystallographic parameters and structural data table are as follows.

TABLE 10
crystal parameters and structural data of crystal form B
Analytical data

| Molecular formula | C₂₉H₃₈ClN₅O₂P•C₇H₇O₃S•H₂O |
|---|---|
| Molecular weight | 744.28 |
| Crystal system | Orthorhombic |
| Space group | P2₁2₁2₁ |
| Cell parameters | a = 7.9891(3) Å, α = 90° |
| | b = 15.7648(7)Å, β = 90° |
| | c = 29.3381(14)Å, γ = 90° |
| Crystal axis ratio | a/b = 0.5068, b/c = 0.5367, c/a = 3.6723 |
| Z | 4 |
| Unit cell volume | 3695.0(3)Å³ |
| Theoretical density | 1.338 Mg/m³ |
| R₁ | 0.050 |
| WR₂ | 0.114 |
| GOOF═S | 1.03 |
| R₍ᵢₙₜ₎ | 0.082 |
| Flackparameter | 0.019(19) |

According to the analysis of X-ray single crystal diffraction data, crystal form B is a monohydrate.

### Example 6 Preparation of crystal form C of mono-p-toluenesulfonate sesquihydrate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (Compound of Formula III)

The crystal form B monohydrate was dissolved in water, prepared into a suspension of about 3 mg/mL and warmed to 50-60° C. to dissolve, the solution was filtered and precipitated at room temperature to obtain the elongated needle-like single crystals, i.e., crystal form C sesquihydrate. The XRPD pattern is shown in FIG. 7 and the data are detailed in Table 11:

TABLE 11
X-ray powder diffraction pattern data of crystal form C

| 2θ (°) | counts | I/I₀ (%) |
|---|---|---|
| 6.0 | 105 | 22.2 |
| 10.8 | 60 | 12.8 |
| 12.1 | 227 | 48.2 |
| 13.4 | 322 | 68.5 |
| 15.0 | 220 | 46.7 |
| 16.9 | 221 | 46.9 |
| 18.4 | 471 | 100.0 |
| 19.0 | 176 | 37.5 |
| 19.3 | 238 | 50.5 |
| 19.8 | 306 | 65.0 |
| 20.9 | 154 | 32.8 |
| 21.8 | 310 | 65.9 |
| 23.2 | 172 | 36.6 |
| 23.6 | 231 | 49.1 |
| 24.3 | 213 | 45.2 |
| 25.5 | 62 | 13.1 |
| 26.6 | 103 | 21.8 |
| 27.7 | 38 | 8.0 |
| 29.7 | 93 | 19.7 |

The crystallographic parameters and structural data of crystal form C single crystal are detailed as described in Table 12;

TABLE 12
crystallographic parameters and structural
data of crystal form C sesquihydrate
Analytical data

| Molecular formula | 2(C₇H₇O₃S)•2(C₂₉H₃₈ClN₅O₂P)•3(H₂O) |
|---|---|
| Molecular weight | 1506.54 |
| Crystal system | Orthorhombic |
| Space group | P2₁2₁2₁ |
| Cell parameters | a = 8.4802(8)Å, α = 90° |
| | b = 15.1272(14)Å, β = 90° |
| | c = 29.429(3)Å, γ = 90° |
| Crystal axis ratio | a/b = 0.5606, b/c = 0.5140, c/a = 3.4703 |
| Z | 2 |
| Unit cell volume | 3775.2(7)Å³ |
| Theoretical density | 1.325 Mg/m³ |
| R₁ | 0.053 |
| WR₂ | 0.114 |
| GOOF═S | 1.06 |
| Analytical data | |
| R₍ᵢₙₜ₎ | 0.041 |
| Flackparameter | 0.029(12) |

From the above single crystal analysis data, it can be determined that the crystal form C is sesquihydrate.

### Example 7 Solubility Related Measurements

Method of solubility test in water for crystal form B of mono-p-toluenesulfonate monohydrate of the compound of formula IV: 0.5 g of crystal form B were accurately weighted, added water dropwise, recorded the amount of solvent added and the dissolution state of crystal form B. When crystal form B was completely dissolved, recorded the amount of solvent added and calculate the critical saturation solubility; if the addition of more than 50 mL of the solvent is still unable to dissolve clear, then take sample centrifugation to detect saturation solubility.

Method of solubility test for crystal form B of mono-p-toluenesulfonate monohydrate of the compound formula IV in various pH values: 0.2 g of crystal form B were accurately weighted, added solvent dropwise, recorded the amount of solvent added and the dissolution state of crystal form B. When crystal form B was completely dissolved, recorded the amount of solvent added and calculate the critical saturation solubility; if the addition of more than 50 mL of solvent is still unable to dissolve clear, then take sample centrifugation to detect saturation solubility. The following table shows the saturated solubility of the compound of formula IV. The solubility of crystal form B of mono-p-toluenesulfonate monohydrate of the compound formula IV is detailed in the following table:

TABLE 13
solubility of crystal form B in various media

| | | Solubility |
|---|---|---|
| pH value | Medium | (mg/mL) |
| pH 1.0 | Dilute hydrochloric acid with water | 87.2 |
| pH 2.0 | Dilute hydrochloric acid with water | 4.7 |
| pH 4.5 | A mixture of sodium acetate trihydrate and glacial | 2 |
| | acetic acid is dissolved in water and diluted | |
| pH 6.0 | A mixture of anhydrous potassium dihydrogen | 1.9 |
| | phosphate and sodium hydroxide is dissolved in | |
| | water and diluted | |
| Water | A mixture of anhydrous potassium dihydrogen | 1.4 |
| | phosphate and sodium hydroxide is dissolved in | |
| | water and diluted | |
| pH 6.8 | A mixture of anhydrous potassium dihydrogen | 2.1 |
| | phosphate and sodium hydroxide is dissolved in | |
| | water and diluted | |
| pH 7.4 | A mixture of anhydrous potassium dihydrogen | 2.1 |
| | phosphate and sodium hydroxide is dissolved in | |
| | water and diluted | |

### Example 8 Preparation of crystal form A of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (Compound of Formula IV)

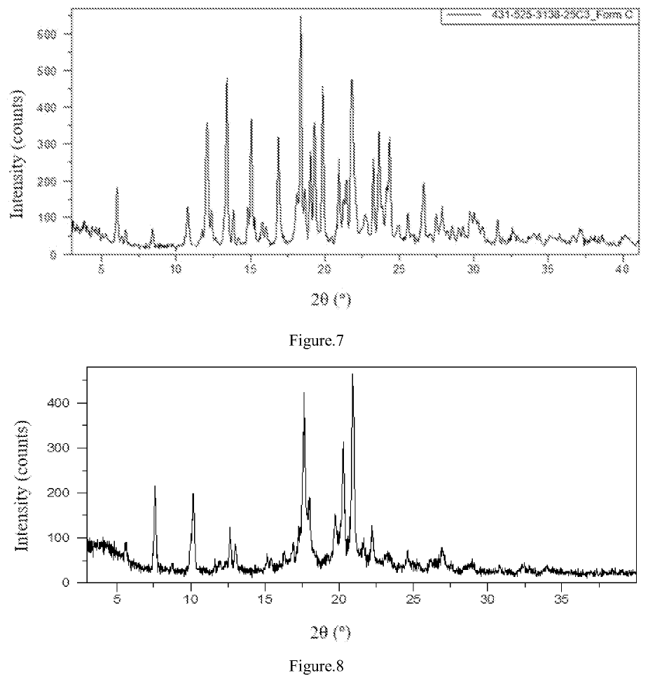

200 mg of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide prepared according to the method of Example 1 was added to a 3 mL glass bottle successively with 2 mL of purified water, and the sample was stirred magnetically for 6 hours at room temperature. After 6 h, the sample was centrifuged and the wet sample was dried under reduced pressure at 40° C. for 21 h to obtain 176 mg of crystal form A in 88.0% yield, whose XRPD pattern is detailed in FIG. 8, and the data of the X-ray powder diffraction pattern is detailed in Table 14.

TABLE 14
X-ray powder diffraction pattern data of crystal form A

| 2θ(°) | Counts | I/I₀ (%) |
|---|---|---|
| 4.1 | 45 | 15.4 |
| 5.6 | 42 | 14.3 |
| 7.6 | 109 | 36.9 |
| 10.2 | 107 | 36.4 |
| 10.9 | 13 | 4.4 |
| 12.6 | 59 | 20.1 |
| 13.0 | 44 | 14.8 |
| 15.2 | 18 | 6.2 |
| 17.6 | 246 | 83.4 |
| 19.7 | 85 | 28.9 |
| 20.3 | 184 | 62.5 |
| 20.9 | 294 | 100.0 |
| 22.2 | 60 | 20.5 |
| 23.2 | 22 | 7.5 |
| 24.6 | 19 | 6.6 |
| 27.0 | 40 | 13.6 |
| 28.8 | 14 | 4.6 |
| 37.0 | 6 | 2.1 |
| 37.7 | 12 | 4.1 |

### Example 9 Physicochemical stability study of crystal form of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide

The free base crystal form A of the compound of formula IV and the crystal form B of mono-p-toluenesulfonate of the compound of formula TV were placed at 40° C./75% RH, 60° C., 75% RH, 92.5% RH and light exposure, respectively, to test the stability, the specific test method was referred to the requirements of the General Rules 9001 for the stability testing of raw pharmaceutical materials and preparations in the 2020 edition of the Chinese Pharmacopoeia. Use XRPD and HPLC to detect the physical and chemical stability of the samples. The detailed results are shown in the table below:

TABLE 15
summary of stability test results of crystal form B

| Placement | Crystal form/purity (A %) | |
|---|---|---|
| conditions | Before placement | After placement |
| 40° C./ | Crystal form B | Crystal form B |
| 75% RH, 5 days | Purity: 99.81 | Purity: 99.81 |
| | Maximum single | Maximum single impurity: 0.04 |
| 40° C./ | impurity: 0.04 | Crystal form B |
| 75% RH, 10 days | | Purity: 99.82 |
| | | Maximum single impurity: 0.04 |
| 40° C./ | | Crystal form B |
| 75% RH, 30 days | | Purity: 99.81 |
| | | Maximum single impurity: 0.04 |
| 60° C., 5 days | | Crystal form B |
| | | Purity: 99.81 |
| | | Maximum single impurity: 0.04 |
| 60° C., 10 days | | Crystal form B |
| | | Purity: 99.82 |
| | | Maximum single impurity: 0.04 |
| 60° C., 30 days | | Crystal form B |
| | | Purity: 99.80 |
| | | Maximum single impurity: 0.04 |
| 75% RH, 5 days | | Crystal form B |
| | | Purity: 99.81 |
| | | Maximum single impurity: 0.04 |
| 75% RH, 10 days | | Crystal form B |
| | | Purity: 99.82 |
| | | Maximum single impurity: 0.04 |
| 75% RH, 30 days | | Crystal form B |
| | | Purity: 99.80 |
| | | Maximum single impurity: 0.04 |
| 92.5% RH, 5 days | | Crystal form B |
| | | Purity: 99.81 |
| | | Maximum single impurity: 0.04 |
| 92.5% RH, 10 days | | Crystal form B |
| | | Purity: 99.82 |
| Light, 5 days | | Maximum single impurity: 0.04 |
| | | Crystal form B |
| | | Purity: 99.67 |
| | | Maximum single impurity: 0.05 |
| Light, 10 days | | Crystal form B |
| | | Purity: 99.60 |
| | | Maximum single impurity: 0.10 |

TABLE 16
stability data of free base crystal form A

| | |
|---|---|
| Placement | Crystal form/purity (A %) |

| conditions | Before placement | After placement |
|---|---|---|
| 60° C., 5 days | Crystal form A | Crystal form A |
| | Purity: 99.33 | Purity: 96.39 |
| | Maximum single | Maximum single |
| | impurity: 0.05 | impurity: 0.75 |
| 60° C., 10 days | | Crystal form A |
| | | Purity: 94.49 |
| | | Maximum single |
| | | impurity: 1.21 |
| 60° C., 30 days | | Not detected |
| | | Purity: 89.65 |
| | | Maximum single |
| | | impurity: 2.02 |

### Example 10 Preparation of salts form and crystal form of other acid of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide (Compound of Formula IV) and Crystal Form Thereof

About 50 mg of the compound of formula IV (prepared according to the method of Example 1) and 1.05 equivalents of acid (hydrochloric acid, with the molar ratio of acid to compound IV set at 2.10) were taken separately, added 1 mL of solvent to the mixture and stirred at room temperature for 2 days. The resulting clarified solution was attempted to crystallize by stirring and slow evaporation at 5° C., and the solid was separated by centrifugation and dried at 40° C. under blast or reduced pressure for 2-5 hours before being used for XRPD characterization.

TABLE 17
salification results of compound of formula IV

| | | Result | |
|---|---|---|---|
| | | | Salification results |
| | | | (molar ratio of |
| | | Salification | compound of formula |
| Acid | Salification solvent | pattern | IV to acid radical) |
| Methane | Toluene | Crystal form | 1:1 |
| sulfonic acid | Ethanol/n-Heptane | | |
| Hydrochloric | Ethanol/n-Heptane | Crystal form | 1:1 |
| acid | Isopropyl alcohol/n-heptane | | |
| | Acetone/n-Heptane | | |
| | Ethyl acetate/n-heptane | | |
| Hydrochloric | Acetone | Crystal form | 1:2 |
| acid | | | |
| Phosphoric acid | Isopropyl alcohol/n-heptane | Not finalized | 1:1 |
| | Acetone/n-heptane | | |
| | Ethyl acetate/n-heptane | | |
| Hippuric acid | Acetone/n-heptane | Crystal form | 1:1 |
| Sulfuric acid | Acetone/n-heptane | Not finalized | 1:1 |
| | Ethyl acetate/n-heptane | | |
| Hydrobromic | Acetone/n-heptane | Crystal form | 1:1 |
| acid | Ethyl acetate/n-heptane | | |
| Benzenesulfonic | Acetone/n-heptane | Crystal form | 1:1 |
| acid | | | |
| Oxalic acid | Ethanol/n-Heptane | Crystal form | 1:1 |
| | Isopropyl alcohol/n-Heptane | | |
| | Acetone/n-Heptane | | |
| Fumaric acid | Acetone/n-Heptane | Crystal form | 1:1 |
| Citric acid | Acetone/n-Heptane | Crystal form | 1:1 |
| Succinic acid | Ethanol/n-Heptane | No salt is produced | |
| | Isopropyl alcohol/n-Heptane | | |
| | Acetone/n-Heptane | | |
| | Ethyl acetate/n-Heptane | | |
| | Acetonitrile-Water/n-Heptane | | |
| Maleic acid | Ethanol/n-Heptane | No salt is produced | |
| | Isopropyl alcohol/n-Heptane | | |
| | Acetone/n-Heptane | | |
| | Ethyl acetate/n-Heptane | | |
| | Acetonitrile-Water/n-Heptane | | |
| Adipic acid | Ethanol/n-Heptane | No salt is produced | |
| | Isopropyl alcohol/n-Heptane | | |
| | Acetone/n-Heptane | | |
| | Ethyl acetate/n-Heptane | | |

| | | |
|---|---|---|
| | Acetonitrile-Water/n-Heptane | |
| L-(+)-tartaric | Ethanol/n-Heptane | No salt is produced |
| acid | Isopropyl alcohol/n-Heptane | |
| | Acetone/n-Heptane | |
| | Ethyl acetate/n-Heptane | |
| | Acetonitrile -Water/n-Heptane | |
| D-glucuronic | Ethanol/n-Heptane | No salt is produced |
| acid | Isopropyl alcohol/n-Heptane | |
| | Acetone/n-Heptane | |
| | Ethyl acetate/n-Heptane | |
| | Acetonitrile-Water/n-Heptane | |
| L-ascorbic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |
| L-malic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |
| Benzoic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |
| Gentisic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |
| L-glutamic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |
| Acetic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |
| stearic acid | Ethanol/n-Heptane | No salt is produced |
| | Isopropyl alcohol/n-Heptane | |
| | Acetone/n-Heptane | |
| | Ethyl acetate/n-Heptane | |
| | Acetonitrile-Water/n-Heptane | |

Specifically, the preparation method for mesylate of the compound of formula IV is as follows:

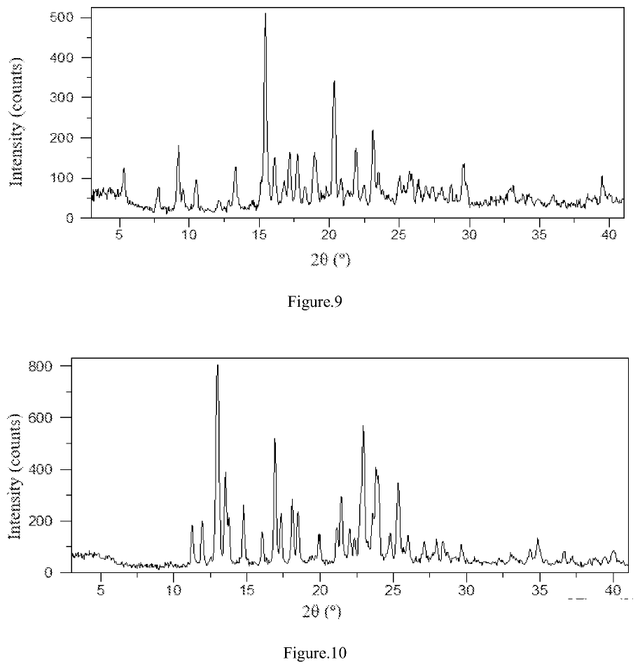

About 50 mg of the compound of formula IV and 1.05 equivalents of methane sulfonic acid were taken separately, 1 mL of toluene were added to the mixture and stirred at room temperature for 2 days. The resulting liquid was then crystallized by slow volatilization under stirring at 5° C. The solid was separated by centrifugation and dried under reduced pressure at 40° C. for 2-5 h to obtain the mesylate crystal form of (S)-(2-((5-chloro-2-((7-(pyrrolidin-1-yl)-6,7,8,9-tetrahydro-5H-benzo[7]annulen-2-yl)amino))-pyrimidin-4-yl)amino)-5-(methoxymethyl)phenyl)dimethyl phosphine oxide with the XRPD as shown in FIG. 9.

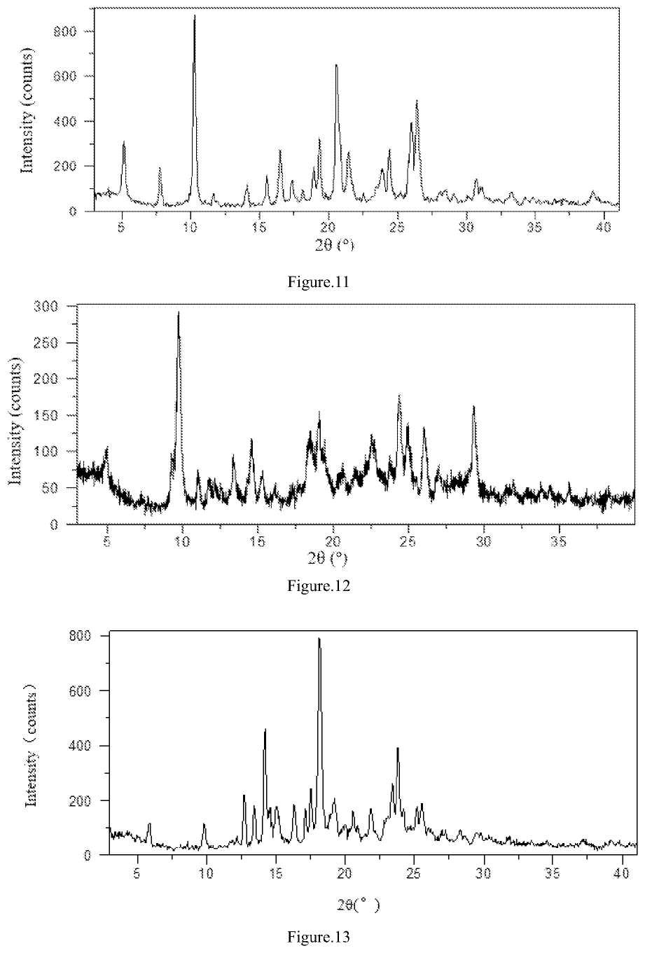

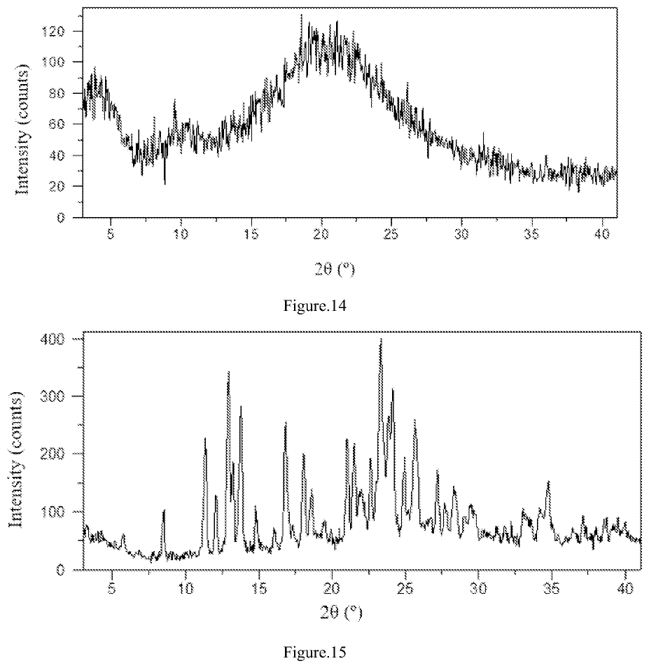

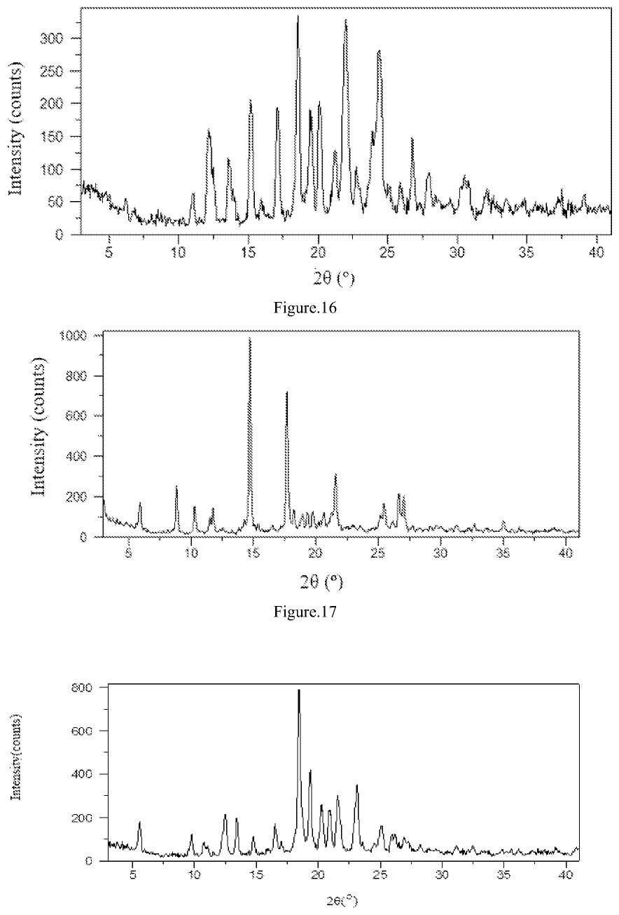

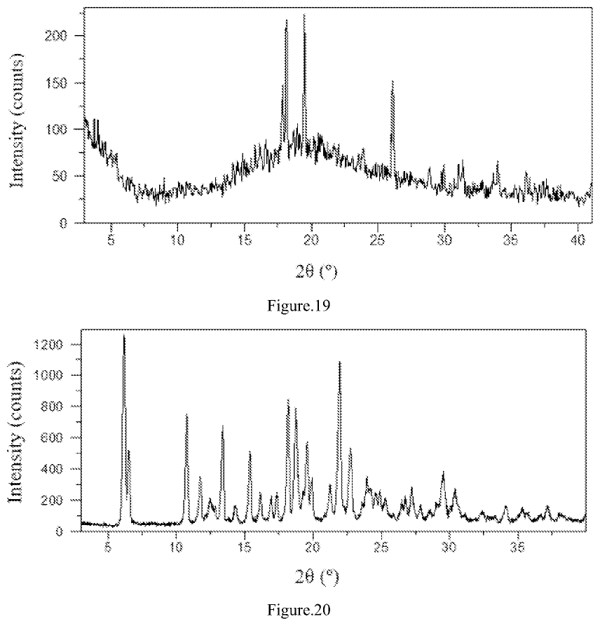

The X-ray powder diffraction (XRPD) spectrum of the hydrochloride, dihydrochloride, phosphate, hippurate, sulfate, hydrobromide, benzenesulfonate, oxalate, fumarate, and citrate of the compound of formula IV are shown in FIG. 10-19, respectively.

### Example 11 Preparation of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or Hydrate Thereof (Compound of Formula I)

The crystal form B prepared in Example 4 was dried under reduced pressure at 40° C. for 16 hours to prepare hydrate of the mono-p-toluenesulfonate of the compound of formula I (X=0˜1), and the sample was taken for XRPD detection, and the X-ray powder diffraction pattern is detailed in FIG. 20.

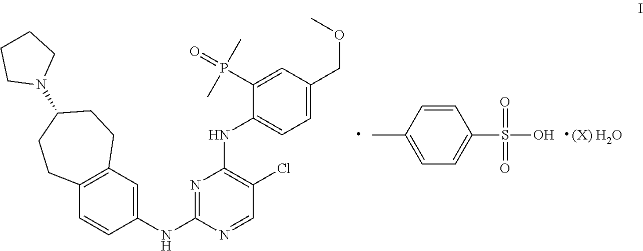

1. A mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or a hydrate thereof.

2. The mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof of claim 1, wherein the specific structure is shown in formula I. 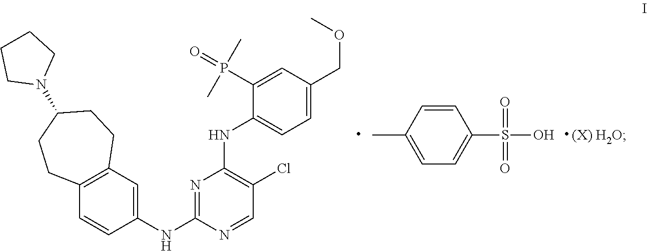
wherein X=0˜2, further, X=0˜1 or 1.5; further, X is 0, 0.25, 0.5, 0.7, 1, 1.25, 1.5 or 1.75.

3. A crystal form of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or a hydrate thereof, wherein the X-ray powder diffraction pattern has diffraction peaks at 2θ of 6.0°±0.2°, 6.3°±0.2°, 10.5°±0.2°, 13.2°±0.2° and 21.8°±0.2°;
further, the X-ray powder diffraction pattern has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 21.8°±0.2° and 22.6°±0.2°;
further, the X-ray powder diffraction pattern has diffraction peaks at 2θ of 6.0°±0.2°, 6.3°±0.2°, 10.5°±0.2°, 11.5°±0.2°, 13.2°±0.2°, 15.2°±0.2°, 18.0°+0.2°, 18.6°+0.2°, 18.7°+0.2°, 19.4°±0.2°, 19.7°±0.2°, 21.8°±0.2°, 22.6°±0.2° and 29.4°±0.2°;
further, the X-ray powder diffraction pattern has diffraction peaks at 2° of 6.0°±0.2°, 6.3°±0.2°, 10.5°±0.2°, 11.5°±0.2°, 12.3°±0.2°, 12.4°±0.2°, 12.6°±0.2°, 13.2°±0.2°, 14.10°±0.2°, 15.2°±0.2°, 15.9°±0.2°, 16.7°±0.2°, 17.1°±0.2°, 18.0°±0.2°, 18.6°±0.2°, 18.7°±0.2°, 19.0°±0.2°, 19.4°±0.2°, 19.7°±0.2°, 21.1°±0.2°, 21.8°±0.2°, 22.6°±0.2°, 23.3°±0.2°, 23.7°±0.2°, 24.1°±0.2°, 24.4°±0.2°, 24.7°±0.2°, 25.1°±0.2°, 26.2°±0.2°, 26.6°±0.2°, 27.0°±0.2°, 27.7°±0.2°, 28.0°±0.2°, 28.3°±0.2°, 28.7°±0.2°, 28.8°±0.2°, 29.4°±0.2°, 30.2°±0.2° and 33.9°±0.02°.

4. The mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof of claim 1, which is a monohydrate, and the specific structure of the monohydrate is shown in formula II:

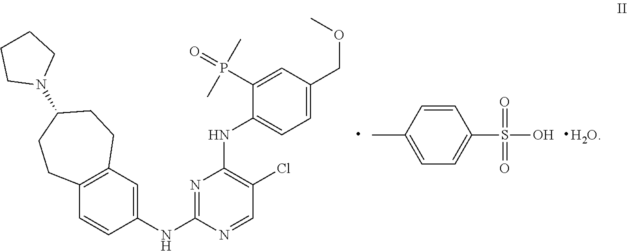

5. The mono-p-toluenesulfonate or hydrate thereof of claim 4, wherein the X-ray powder diffraction pattern of the monohydrate crystal form has diffraction peaks at 2θ of 6.0°±0.2°, 6.3°±0.2°, 10.5°±0.2°, 13.2°±0.2° and 21.8°±0.2°;
further, the X-ray powder diffraction pattern has diffraction peaks at 2θ of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 21.8°±0.2°, and 22.6°±0.2°;
further, the X-ray powder diffraction pattern has diffraction peaks at 2° of 6.0°±0.2°, 6.3°±0.2°, 10.5°±0.2°, 11.5°±0.2°, 13.2°±0.2°, 15.2°±0.2°, 18.0°±0.2°, 18.6°±0.2°, 18.7°±0.2°, 19.4°±0.2°, 19.7°±0.2°, 21.8°±0.2°, 22.6°±0.2° and 29.4°±0.2°;
further, the X-ray powder diffraction pattern has diffraction peaks at 2θ of 6.0°±0.2°, 6.3°±0.2°, 10.5°±0.2°, 11.5°±0.2°, 12.3°±0.2°, 12.4°±0.2°, 12.6°±0.2°, 13.2°±0.2°, 14.1°±0.2°, 15.2°±0.2°, 15.9°±0.2°, 16.7°±0.2°, 17.1°±0.2°, 18.0°±0.2°, 18.6°±0.2°, 18.7°±0.2°, 19.0°±0.2°, 19.4°±0.2°, 19.7°±0.2°, 21.1°±0.2°, 21.8°±0.2°, 22.6°±0.2°, 23.3°±0.2°, 23.7°±0.2°, 24.1°±0.2°, 24.4°±0.2°, 24.7°±0.2°, 25.1°±0.2°, 26.2°±0.2°, 26.6°±0.2°, 27.0°±0.2°, 27.7°±0.2°, 28.0°±0.2°, 28.3°±0.2°, 28.7°±0.2°, 28.8°±0.2°, 29.4°±0.2°, 30.2°±0.20 and 33.9°±0.2°;
further, the X-ray powder diffraction expressed in an angle of 20 has a pattern shown in FIG. 1.

6. The mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof of claim 1, which is a sesquihydrate, and the specific structure of the sesquihydrate is shown in formula III:

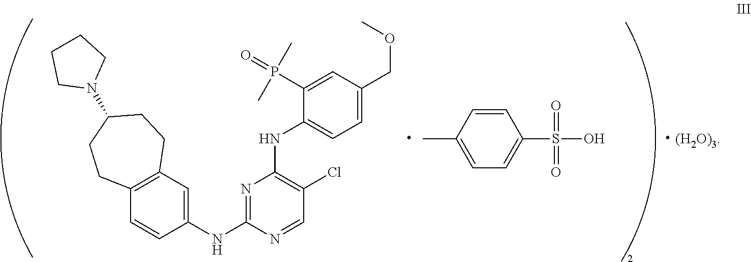

7. The mono-p-toluenesulfonate or hydrate thereof of claim 6, wherein the X-ray powder diffraction pattern of the sesquihydrate crystal form has diffraction peaks at 2θ of 13.4°±0.2°, 18.4°±0.2°, 19.3°±0.2°, 19.8°±0.2° and 21.8°±0.2°;
further, the X-ray powder diffraction pattern has diffraction peaks at 2° of 12.1°±0.2°, 13.4°±0.2°, 15.0°±0.2°, 16.9°±0.2°, 18.4°±0.2°, 19.3°±0.2°, 19.8°±0.2°, 21.8°±0.2°, 23.6°±0.2° and 24.30° 0.2°;
further, the X-ray powder diffraction pattern has diffraction peaks at 2θ of 6°±0.2°, 10.8°±0.2°, 12.1°±0.2°, 13.4°±0.2°, 15.0°±0.2°, 16.9°±0.2°, 18.4°±0.2°, 19.0°±0.2°, 19.30° 0.2°, 19.8°±0.2°, 20.9°±0.2°, 21.8°±0.2°, 23.2°±0.2°, 23.6°±0.2°, 24.3°±0.2°, 25.5°±0.2° and 26.6°±0.2°;
further, the X-ray powder diffraction expressed in an angle of 20 has a pattern shown in FIG. 7.

8. A method for preparing the mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof of claim 1, comprising the step of satisfying the compound of formula IV with p-toluenesulfonic acid, the compound of formula IV having the following structure.

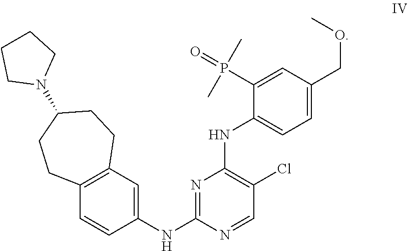 [SMILES: COCc1ccc(Nc2nc(Nc3ccc4c(c3)CC[C@@H](N3CCCC3)CC4)ncc2Cl)c(P(C)(C)=O)c1]

9. A crystal form composition, wherein the mono-p-toluenesulfonate or hydrate thereof of claim 1 constitutes more than 50% by weight of the crystal form composition.
further, the mono-p-toluenesulfonate or hydrate thereof constitutes more than 80% by weight of the crystal form composition
further, the mono-p-toluenesulfonate or hydrate thereof constitutes more than 90% by weight of the crystal form composition;
further, the mono-p-toluenesulfonate or hydrate thereof constitutes more than 95% by weight of the crystal form composition.

10. A pharmaceutical composition of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, comprising the mono-p-toluenesulfonate or hydrate thereof of claim 1.

11. A method for preventing and/or treating AXL kinase-mediated diseases or disease states, comprising administering to an individual in need thereof the mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof claim 1 or a pharmaceutical composition comprising a therapeutically effective amount of the mono-p-toluenesulfonate or hydrate thereof of claim 1.

12. A crystal form composition, wherein the mono-p-toluenesulfonate or hydrate thereof of claim 2 constitutes more than 50% by weight of the crystal form composition;
further, the mono-p-toluenesulfonate or hydrate thereof constitutes more than 80% by weight of the crystal form composition;
further, the mono-p-toluenesulfonate or hydrate thereof constitutes more than 90% by weight of the crystal form composition;
further, the mono-p-toluenesulfonate or hydrate thereof constitutes more than 95% by weight of the crystal form composition.

13. A crystal form composition, wherein the mono-p-toluenesulfonate or hydrate thereof of claim 3 constitutes more than 50% by weight of the crystal form composition;
further, the mono-p-toluenesulfonate or hydrate thereof constitutes more than 80% by weight of the crystal form composition;
further, the mono-p-toluenesulfonate or hydrate thereof constitutes more than 90% by weight of the crystal form composition;
further, the mono-p-toluenesulfonate or hydrate thereof constitutes more than 95% by weight of the crystal form composition.

14. A pharmaceutical composition of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, comprising the mono-p-toluenesulfonate or hydrate thereof of claim 2.

15. A pharmaceutical composition of mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide, comprising the mono-p-toluenesulfonate or hydrate thereof of claim 3.

16. A method for preventing and/or treating AXL kinase-mediated diseases or disease states, comprising administering to an individual in need thereof the mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof of any one of claim 2, or a pharmaceutical composition, the pharmaceutical composition comprising a therapeutically effective amount of mono-p-toluenesulfonate or hydrate thereof of claim 2.

17. A method for preventing and/or treating AXL kinase-mediated diseases or disease states, comprising administering to an individual in need thereof the mono-p-toluenesulfonate of (S)-(2-((5-chloro-2-((7-(pyrrolidin-1-yl)-6,7,8,9-tetrahydro-5H-benzo[7]annulen-2-yl)amino)pyrimidin-4-yl)amino)-5-(methoxymethyl)phenyl)dimethylphosphine oxide or hydrate thereof of any one of claim 3, or a pharmaceutical composition, the pharmaceutical composition comprising a therapeutically effective amount of mono-p-toluenesulfonate or hydrate thereof of claim 3.
